SEQUENCE LISTING (6 sequences)

SEQ ID NO: 1 - protein, 8 aa, Homo sapiens
  MOD_RES (8)..(8): AMIDATION
YFLFRPRN

SEQ ID NO: 2 - protein, 8 aa, Artificial Sequence
  variant NMU-8
YXLFRPRN

SEQ ID NO: 3 - protein, 8 aa, Artificial Sequence
  variant NMU-8
YXLFRPRN

SEQ ID NO: 4 - protein, 8 aa, Artificial Sequence
  variant NMU-8
YXLFRARN

SEQ ID NO: 5 - protein, 8 aa, Artificial Sequence
  variant NMU-8
YXLFRXRN

SEQ ID NO: 6 - protein, 8 aa, Artificial Sequence
  variant NMU-8
YXLXRPRN

(19) United States
(12) Patent Application Publication
(10) Pub. No.: US 2012/0094898 A1
(43) Pub. Date: Apr. 19, 2012

(54) PEPTIDE DERIVATIVE

(75) Inventors: Taiji Asami, Fujisawa (JP); Naoki Nishizawa, Fujisawa (JP); Yoko Kanematsu, Fujisawa (JP)

(73) Assignee: Takeda Pharmaceutical Company Limited, Osaka-Shi (JP)

(21) Appl. No.: 13/271,763

(22) Filed: Oct. 12, 2011

(30) Foreign Application Priority Data
Oct. 13, 2010 (JP) 2010-231016

(51) Int. Cl. A61K 38/08; A61P 3/04; C07K 7/06

(52) U.S. Cl. 514/4.9; 530/328; 514/4.8

(57) ABSTRACT

The present invention relates to a peptide derivative selected from the group consisting of
PEG20k(AL)-β-Ala-Tyr-Nal(1)-Leu-Phe-Arg-Pro-Arg-Asn-NH₂,
PEG20k(AL)-β-Ala-Tyr-Nal(2)-Leu-Phe-Arg-Pro-Arg-Asn-NH₂,
PEG20k(AL)-NpipAc-Tyr-Nal(2)-Leu-Phe-Arg-Pro-Arg-Asn-NH₂,
PEG20k(AL)-NpipAc-Tyr-Nal(2)-Leu-Phe-Arg-Ala-Arg-Asn-NH₂,
PEG20k(AL)-PEG(2)-Tyr-Nal(2)-Leu-Phe-Arg-NMeAla-Arg-Asn-NH₂,
PEG20k(AL)-Pic(4)-Tyr-Nal(2)-Leu-Phe-Arg-NMeAla-Arg-Asn-NH₂,
PEG20k(AL)-Acp-Tyr-Nal(2)-Leu-Phe-Arg-NMeAla-Arg-Asn-NH₂, and
PEG20k(AL)-β-Ala-Tyr-Nal(2)-Leu-Pya(4)-Arg-Pro-Arg-Asn-NH₂;
   or a salt thereof.

### CROSS-REFERENCE TO RELATED APPLICATIONS

This application claims priority to Japanese Patent Application No. 2010-231016, filed Oct. 13, 2010. The entire contents of the aforementioned patent application is incorporated herein by this reference.

### TECHNICAL FIELD

The present invention relates to peptide derivatives.

### BACKGROUND OF INVENTION

Neuromedin U (hereinafter sometimes referred to as “NMU”) was first isolated, as a peptide consisting of 25 amino acid residues or as a peptide consisting of 8 amino acid residues, from the pig small intestine using uterine smooth muscle contraction activity as an index. These peptides are named porcine NMU-25 or porcine NMU-8, based on the number of amino acid residues. Porcine NMU-8 is a cleavage product of porcine NMU-25 and consists of the C-terminal 8 residues of porcine NMU25.

Similarly, NMU-25 is known in humans. The amino acid sequence of the C-terminal 8 residues of human NMU-25 is the same as that of the C-terminal 8 residues of porcine NMU-8.

Rat NMU consists of 23 amino acid residues, and is named rat NMU-23. The amino acid sequence of the C-terminal 8 residues of rat NMU-23 differs from that of the C-terminal 8 residues of porcine NMU-8 by one amino acid residue.

As a receptor for NMU, FM3, which is an orphan GPCR, was initially identified; subsequently, TGR1 was identified. Today, these receptors are called NMUR1 and NMUR2, respectively. FM3 is primarily distributed in the intestinal tract, whereas TGR1 is localized in the hypothalamus.

As a ligand for TGR1, a novel peptide has been isolated from rat brain. Since this peptide is localized in the suprachiasmatic nucleus within the hypothalamus, it was named neuromedin S (NMS), using the initial letter of the suprachiasmatic nucleus.

Human NMS consists of 33 amino acid residues, and the amino acid sequence of the C-terminal 8 amino acid residues are the same as the amino acid sequence of the C-terminal 8 residues of rat NMU-23.

NMUR1 and NMUR2 exhibit similar affinity to NMU, NMS, and NMU-8. It has been suggested that these receptors strongly recognize the amino acid sequence of the C-terminal 8 residues, the sequence of which is common to NMU and NMS.

An intraventricular administration of rat NMU-23 in rats attenuates food intake. A local injection of NMU to the paraventricular nucleus (PVN) or arcuate nucleus (ARC) has also been reported to exhibit an anorectic activity as in the case of its intraventricular administration; therefore, the action sites of NMU are assumed to be PVN and ARC. Further, an intraventricular administration of anti-NMU antibody has shown to increase food intake, suggesting that the central NMU produces physiological effects that suppress food intake. It has also been reported that NMU KO mice exhibited an obese phenotype, and that mice over-expressing NMU exhibited lower body weight and reduced food intake. This clarifies the physiological significance of endogenous NMU (Nature, 406, pp. 70-74, 2000).

It has further been reported that an intraventricular administration of NMU causes an elevation of body temperature, generation of heat, and elevation of oxygen consumption. These activities are assumed to be due to sympathetic activation of adipose tissue and muscle system.

It has also been reported that suppression of gastric acid secretion and suppression of gastric emptying are caused by an intraventricular administration of NMU. These activities are assumed to be due to the central effects via CRH secretion. These activities result in reduced food intake.

It has not yet been examined in detail how a peripheral administration of NMU causes an action on the intestinal tract; however, considering that NMUR1 is expressed in the intestinal tract, it can be assumed that the peripheral administration of NMU causes a certain action on the intestinal tract. Based on this assumption, action on the stomach or intestinal tract caused by NMU peripheral administration was examined, and colon-specific prokinetic activity has been discovered.

WO 2007/075439 and WO 2007/109135 disclose that an anorectic effect is achieved by peripheral administration of NMU. WO 2010/053830 discloses that a peripheral administration of NMUR agonist accelerates the secretion of GLP-1 and Peptide YY (PYY).

WO 2007/075439 discloses a following compound:

An polypeptide comprising an FNX Peptide, wherein the FNX Peptide comprises an amino acid sequence of formula (I): F1-P, where F1-P is a combination of an F1 segment and a P segment, where P is an octapeptide capable of providing, when attached to F1 and systemically delivered, suppression of food intake, reduction of body weight, and/or induction of a satiety signal or a distension signal, and wherein F1 is a des-octapeptide portion of an FN38 or analog, derivative or chimera thereof, which enhances or enables P activity, and with the proviso that excluded from F1-P are the polypeptides corresponding to GenBank Accession Number AJ510133 (human), CAD52851 (rat), CAD52850 (frog) and chicken FN38.

WO 2007/109135 discloses a neuromedin U receptor agonist, which has the formula:

Z¹-peptide-Z²
wherein the peptide has the amino acid sequence X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—X¹⁸—X¹⁹—X²⁰—X²¹—X²²—X²³—X²⁴—X²⁵ (SEQ ID NO.27), wherein amino acids 1 to 17 can be any amino acid or absent; wherein amino acid X¹⁸ is absent, Y, W, F, a des-amino acid or an acyl group; amino acid X¹⁹ is A, W, Y, F or an aliphatic amino acid; amino acid X²⁰ is absent, L, G, sarcosine (Sar), D-Leu, NMe-Leu, D-Ala or A; amino acid X²¹ is F, NMe-Phe, an aliphatic amino acid, an aromatic amino acid, A or W; X²² is R, K, A or L; amino acid X²³ is F, Sar, A or L; amino acid X²⁴ is R, Harg or K; and amino acid X²⁵ is N, any O- or L-amino acid, Nle or D-Nle, A; and Z¹ is an optionally present protecting group that, if present, is joined to the N-terminal amino group; and Z² is NH₂ or an optionally present protecting group that, if present, is joined to the C-terminal carboxy group, and pharmaceutically acceptable salts thereof.

WO 2010/053830 discloses a following method:

A method of determining the efficacy of a composition comprising a neuromedin U receptor agonist given to an individual for the treatment of a metabolic disorder, comprising:
(a) assaying a plasma sample from the individual to determine a level of glucagon-like peptide 1 (GLP-1) and/or peptide YY (PYY) at a first time point;
(b) administering the composition to the individual; and
(c) thereafter assaying a plasma sample from the individual to determine the level of GLP-1 and/or PYY at a second time point;
wherein an increased level of GLP-I and/or PYY at the second time point relative to the first time point is indicative of the efficacy of the composition in treating the metabolic disorder.

WO 2009/044918 discloses a following compound:

A neuromedin U derivative
which is a polypeptide consisting of an amino acid sequence which is bound with a methoxypolyethylene glycol(s) via a linker,

said amino acid sequence contains at least 8 amino acids of the C-terminus of an amino acid sequence of neuromedin U, and is the same or substantially the same as the amino acid sequence of neuromedin U, and

which is represented by a formula:

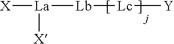

wherein

Y represents a polypeptide consisting of a amino acid sequence which contains at least 8 amino acids of the C-terminus of neuromedin U and is the same or substantially the same as the amino acid sequence of neuromedin U;

X represents a methoxyethylene glycol;

X′ is absent or represents a methoxypolyethylene glycol;

the part represented by a formula (II):

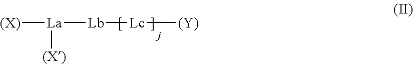

represents a linker,

La represents a divalent or trivalent group selected from

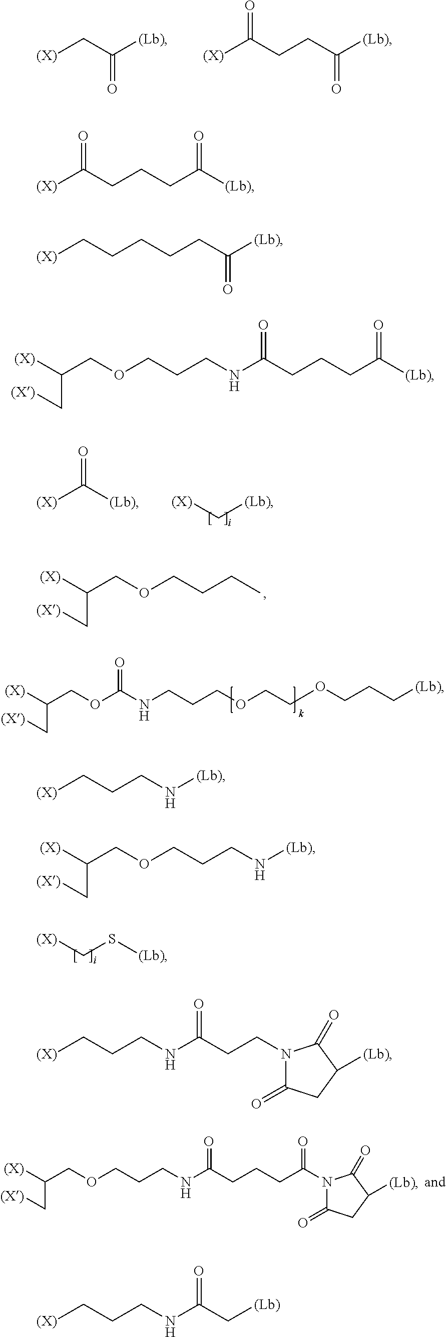

wherein
i represents an integer ranging from 1 to 5 and
k represents an integer ranging from 1 to 100;

Lb represents

(i) a bond,

(ii) a divalent group represented by a formula:
wherein
B^{1a} and B^{1b} represent —CO—,
Q^{b1} represents a divalent group selected from

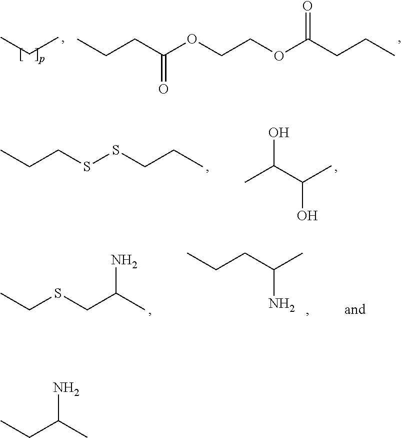

wherein p represents an integer ranging from 2 to 8,

(iii) a divalent group represented by a formula: —B^{2a}-Q^{b2}-B^{2b}—
wherein
B^{2a} represents —CO—,
B^{2b} represents

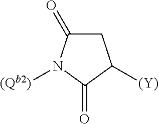

Q^{b2} represents a divalent group selected from

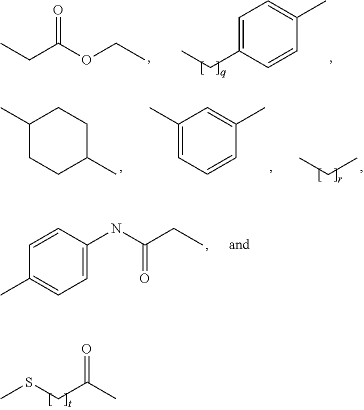

wherein
q represents an integer ranging from 3 to 10,
r represents an integer ranging from 1 to 10, and
t represents an integer ranging from 1 to 10, or

(iv) a divalent group represented by a formula: —B^{3a}-Q^{b3}-B^{3b}—
wherein
B^{3a} represents

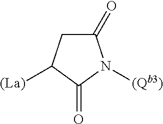

or a bond,
B^{3b} represents —CO—,
Q^{b3} resents a divalent group represented by a formula: —(CH₂)ₙ₁—Z—(CH₂)ₙ₂—
wherein n1 represents an integer ranging from 0 to 5,
n2 represents an integer ranging from 0 to 5,
Z represents a bond, —O—CO—, —CO—NH—, —CO—O—, —NH—CO—,

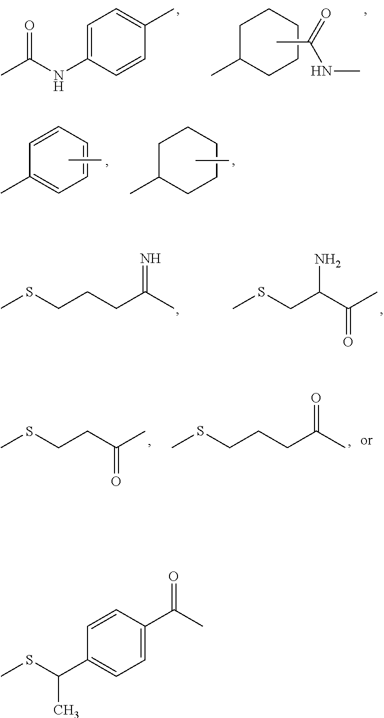

Lc represents

(i) a divalent group represented by a formula:
wherein
C^{a} represents —NH—,
Q^{c} represents a divalent group of a formula: —(CH₂)ₘ₁—Z^{c}—(CH₂)ₘ₂—
   wherein
   m1 represents an integer ranging from 0 to 15,
   Z^{c} represents
   (a) a bond or
   (b) a divalent group selected from —CO—, —O—CO—, —CO—O—, —CO—NH—, —NH—CO—, —CO—NH—CO—, —NH—CO—NH—, —CH(NH₂)—, —CH(—NHR^{Zc1})—, —CH(R^{Zc2})—, —CH(OH)—, —CH(COOH)—, —C(═NH)—, —CH(—NHX)—,

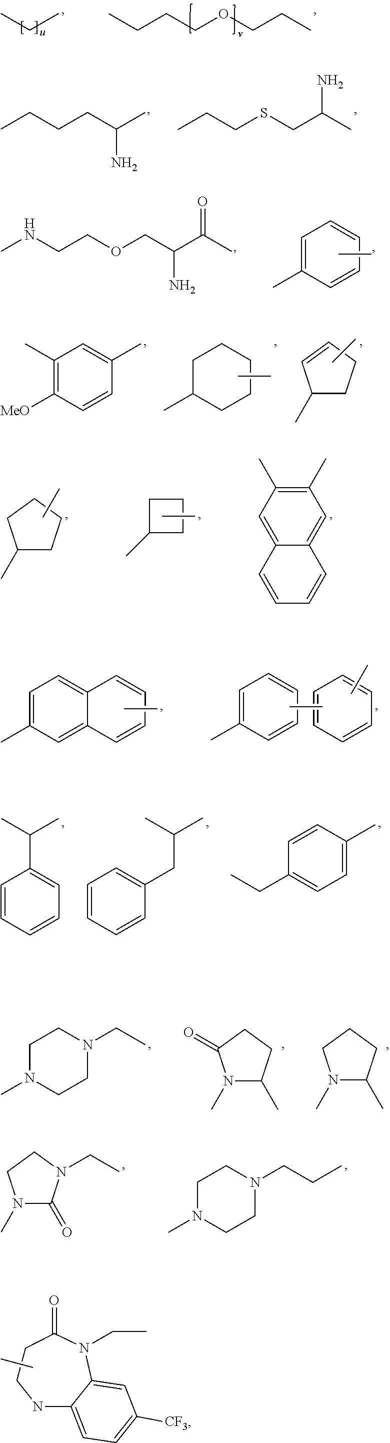

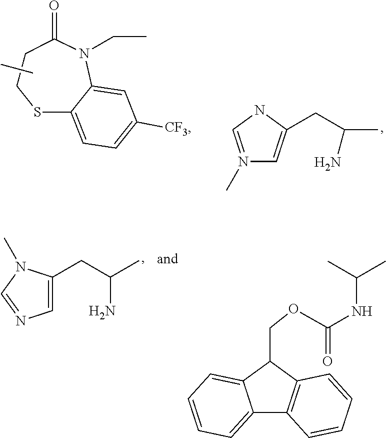

wherein
u represents an integer ranging from 1 to 18,
v represents an integer ranging from 1 to 12,
R^{Zc1} represents an amino-straight chain C₁₋₅ alkyl-carbonyl group or X-straight chain C₁₋₅ alkyl group,
R^{Zc2} represents an amino-straight chain C₁₋₅ alkyl-carbonyl amino-straight chain C₃₋₅ alkyl group, and
X represents the same as mentioned above, and
m2 represents an integer ranging from 0 to 15, and
C″ represents a bond, —CO—, or —SO₂—, or

(ii) a divalent group represented by a formula: -Q^{c′}-C^{b′}—
wherein
Q^{c′} represents a divalent group represented by a formula: —(CH₂)_{m1′}—Z^{c′}—(CH₂)_{m2′}—
   wherein
   m1′ represents an integer ranging from 0 to 15,
   Z^{c′} represents

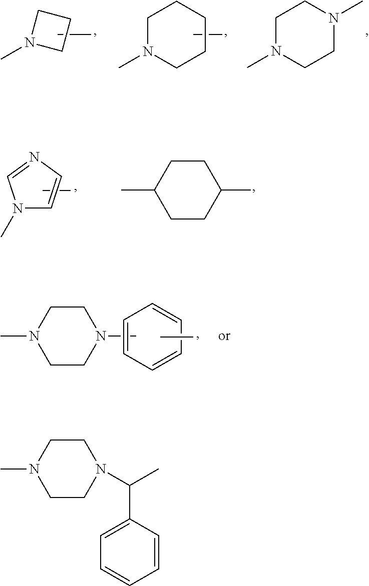

and
m2′ represents an integer ranging from 0 to 15,
C^{b′} represents —CO— or —SO₂—;

j represents an integer ranging from 0 to 3,

provided that, if La is

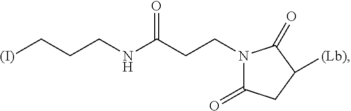

and Lb is a bond,
then Lc is not a bond; and
further provided that

### if La is

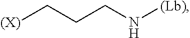

and
Lb is a divalent group represented by a formula: —CO-Q^{b2}-B^{2b}—

wherein

Q^{b2} is

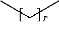

wherein r is 2,

B^{2b} is

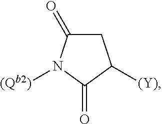

then Lc is not a bond.

WO 2010/116752 discloses a compound represented by the following formula (I):

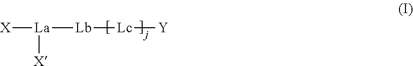

[wherein X represents a polypeptide consisting of an amino acid sequence set forth in SEQ ID NO.: 1 wherein 1 to 4 amino acids are substituted,
the amino acid substitution is selected from:
(1) substitution of Tyr at position 1 with Ala, Arg, Glu, Ser, Gln, NMeArg, Phe, NMeTyr, D-Tyr, Trp, or Pro;
(2) substitution of Phe at position 2 with Val, Gln, Arg, Glu, Set, Tyr, Pro, Cha, Trp, NMePhe, Nle, Tyr(PO₃H₂), Hse, Nal(1), Nal(2), Phe(4F), or Aib;
(3) substitution of Leu at position 3 with Gln, Arg, Glu, Ser, Val, Phe, Pro, Thr, Cha, Nle, NMeArg, Ile, Leu(Me), Lys, NMeLeu, D-Leu, Ala, D-Ala, Gly, Abu, or Aib;
(4) substitution of Phe at position 4 with Gln, Leu, Pro, Cha, NMePhe, Trp, Phe(4F), Pya(4), αMePhe, Nle, Ala, or Aib;
(5) substitution of Arg at position 5 with Nle, Gln, NMeArg, Orn, Dbu, Pya(4), Hse, or Aib;
(6) substitution of Pro at position 6 with Ala, Hyp, NMeAla, MeGly, NMeSer, D-NMeAla, or Aib;
(7) substitution of Arg at position 7 with Arg(Me) or NMeArg; and
(8) substitution of Asn at position 8 with Nle, Gln, Arg, Asp, Pro, Abu, NMeAsn, or Aib;
X represents a methoxypolyethylene glycol;
X′ is absent or represents a methoxypolyethylene glycol;
La is a divalent or trivalent group represented by formula

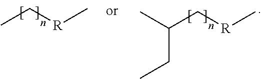

(wherein R represents a bond, —O—, —CO—O—, —O—CO—, —NH—, —CO—, —S—, —S—S—, —SO—, —SO₂—, —NH—SO₂—, —SO₂—NH—, —C(═O)—NH—N═CH—, —C(═NH)—NH—, —CO—CH₂—S—, or

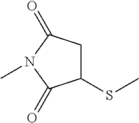

and
n is an integer of 0 to 5);
Lb represents —(CH₂)ᵢ— (wherein i is an integer of 1 to 5);
Lc is a divalent group represented by formula (I): —NH-Q^{c}-C^{b}—

(wherein Q^{c} is a divalent group represented by formula: —(CH₂)ₘ₁—Z^{c}—(CH₂)ₘ₂—

(wherein m1 is an integer of 0 to 15,
Z^{c} represents (a) a bond or (b) a divalent group selected from —CO—, —O—CO—, —CO—O—, —CO—NH—, —NH—CO—, —CO—NH—CO—, —NH—CO—NH—, —CH(NH₂)—, —CH(—NHR^{zc1})—, —CH(R^{zc2})—, —CH(OH)—, —CH(COOH)— —C(═NH)—, —S—, —S—S—, —SO—, —SO₂—, —NH—SO₂—, —SO₂—NH—,

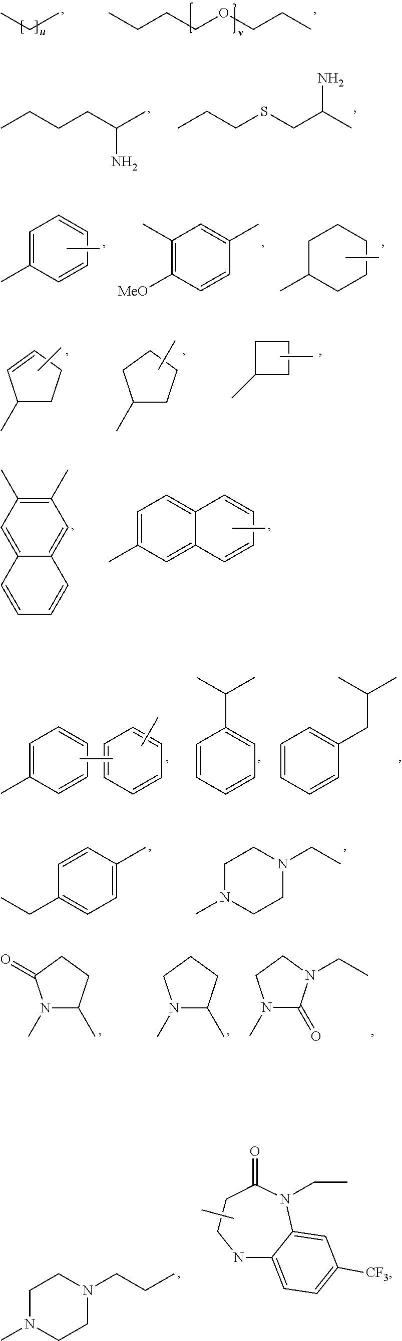

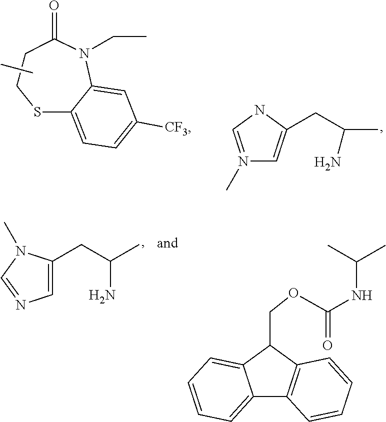

(wherein u is an integer of 1 to 18,

v is an integer of 1 to 12,

R^{zc1} represents an amino-straight chain C₁₋₅ alkyl-carbonyl group, or an X-straight chain C₁₋₅ alkyl group (wherein X is as defined above), and

R^{zc2} represents an amino-straight chain C₁₋₅ alkyl-carbonylamino-straight chain C₁₋₅ alkyl group), and

m2 is an integer of 0 to 15), and

C^{b} represents a bond, —CO—, or —SO₂—), or

a divalent group represented by formula (ii): -Q^{c′}-C^{b′}—
(wherein Q^{c′} represents a divalent group selected from formula: —(CH₂)_{m1′}—Z^{c′}—(CH₂)_{m2′}—
(wherein m1′ is an integer of 0 to 15,

Z^{c′} represents a divalent group selected from

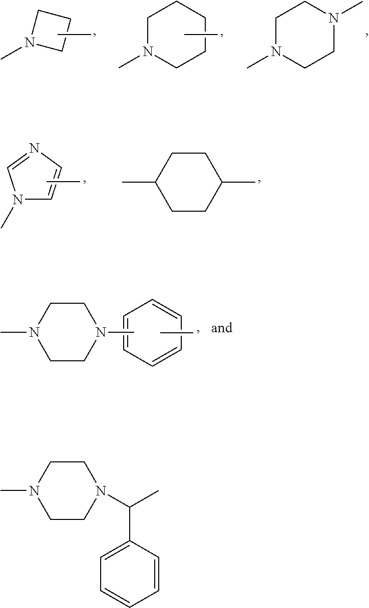

and m2′ is an integer of 0 to 15), and

C^{b′} represents —CO— or —SO₂—; and

j is an integer of 1 to 3]; or
a salt thereof.

WO 2011/005611 discloses a following composition:

A composition comprising the formula
Z¹-peptide-Z²
wherein the peptide has the amino acid sequence X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—X¹⁸—X¹⁹—X²⁰—X²¹—X²²—X²³—X²⁴—X²⁵ (SEQ ID NO:1), wherein amino acids 1 to 17 can be any amino acid or absent; wherein amino acid X¹⁸ is absent, Tyr or D-Tyr, Leu, Phe, Val, Gln, Nle, Glu or D-Glu, Asp, Ala, D-Lys, an aromatic amino acid, a des-amino acid or an acyl group; amino acid X¹⁹ is Ala, Trp, Tyr, Phe, Glu, Nva, Nle or an aromatic amino acid; amino acid X²⁰ is absent, Leu, Gly, sarcosine (Sar), D-Leu, NMe-Leu, D-Ala or Ala, or any D- or L-amino acid; amino acid X²¹ is Phe, NMe-Phe, an aliphatic amino acid, an aromatic amino acid, Ala or Trp; X²² is Arg, Lys, Harg, Ala, or Leu; amino acid X²³ is Pro, Ser, Sar, Ala or Leu; amino acid X²⁴ is Arg, Harg or Lys; and amino acid X²⁵ is Asn, any D- or L-amino acid, Nle or D—Nle, D-Ala or Ala; Z¹ is optionally a protecting group that, if present, is joined to the N-terminus amino group; and
Z² is NH₂ or an optionally present protecting group that, if present, is joined to the C-terminal carboxy group, and pharmaceutically acceptable salts thereof.

WO 2010/138343 discloses a following composition:

A composition comprising a neuromedin U receptor agonist in which neuromedin U or an analog thereof is conjugated to cysteine residue 34 of human serum albumin by a non-maleimido or non-succinimidyl linkage or a pharmaceutically acceptable salt thereof.

WO 2009/042053 discloses a neuromedin U receptor agonist represented by the following formula:

Z¹-peptide-Z²
wherein the peptide has the amino acid sequence ILQRG SGTAA VDFTK KDHTA TWGRP FFLFR PRN (SEQ ID NO: 1), wherein the peptide can have one or more insertions or substitutions of the amino acid sequence with an alternative amino acid and wherein the peptide can have one or more deletions of the amino acid sequence; Z¹ is an optionally present protecting group that, if present, is joined to the N-terminal amino group; and Z² is NH₂ or an optionally present protecting group that, if present, is joined to the C-terminal carboxy group; and pharmaceutically acceptable salts thereof.

Structure-Activity Relationships of Neuromedin U. III. Contribution of Two Phenylalanine Residues in Dog Neuromedin U-8 to the Contractile Activity is disclosed in Chemical & Pharmaceutical Bulletin 1996, 44(10), p. 1880-1884.

### SUMMARY OF INVENTION

### Problems to be Solved by the Invention

Rat NMU exhibits an anorectic effect when administered peripherally. In contrast, although NMU-8 has a sufficiently strong agonist activity to the receptor NMUR1 and NMUR2, NMU-8 does not exhibit an anorectic effect when administered peripherally. It is very important that neuromedin U exhibits a high anorectic effect even when administered in a usual manner so that neuromedin U can be useful as an anorectic agent, for example, peripherally. Thus, an object of the present invention is to provide a peptide derivative, more specifically, a neuromedin U derivative that exhibits a high anorectic effect even when administered in a usual manner, for example, peripherally. Another object of the present invention is to provide a novel agent for preventing or treating obesity, etc., or an anorectic agent.

### Solution to be Solved by the Invention

The inventors of the present invention hypothesized that a cause for the absence of anorectic activity upon peripheral administration is instability of the NMU-8 in the blood. Further, the inventors inferred that a NMU-8 derivative (or a modified compound thereof) that is highly stable in the blood exhibits a sufficient anorectic activity.

Thus, the inventors prepared a peptide derivative (specifically, a neuromedin U derivative) comprising a specific polypeptide which is produced by introducing substitution of 1 or more amino acid residues into an amino acid sequence consisting of 8 amino acids of the C-terminus of neuromedin U, and to which PEG20k (AL) is bound via a linker. The inventors revealed that such a peptide derivative exhibits a sufficiently strong anorectic effect and bodyweight reducing effect even when administered peripherally.

The inventors found that a peptide derivative, which is a compound or a salt thereof as defined below in [1] (hereinafter sometimes referred to as “compound (I)”) is a neuromedin U receptor agonist, and exhibits excellent effects as an agent for preventing or treating obesity, etc. Based on this finding, the inventors diligently carried out further research, and completed the present invention.

More specifically, the present invention provides the following:

[1] A peptide derivative selected from the group consisting of
PEG20k(AL)-β-Ala-Tyr-Nal(1)-Leu-Phe-Arg-Pro-Arg-Asn-NH₂,
PEG20k(AL)-β-Ala-Tyr-Nal(2)-Leu-Phe-Arg-Pro-Arg-Asn-NH₂,
PEG20k(AL)-NpipAc-Tyr-Nal(2)-Leu-Phe-Arg-Pro-Arg-Asn-NH₂,
PEG20k(AL)-NpipAc-Tyr-Nal(2)-Leu-Phe-Arg-Ala-Arg-Asn-NH₂.
PEG20k(AL)-PEG(2)-Tyr-Nal(2)-Leu-Phe-Arg-NMeAla-Arg-Asn-NH₂,
PEG20k(AL)-Pic(4)-Tyr-Nal(2)-Leu-Phe-Arg-NMeAla-Arg-Asn-NH₂,
PEG20k(AL)-Acp-Tyr-Nal(2)-Leu-Phe-Arg-NMeAla-Arg-Asn-NH₂, and
PEG20k(AL)-β-Ala-Tyr-Nal(2)-Leu-Pya(4)-Arg-Pro-Arg-Asn-NH₂, or a salt thereof;
[1A] PEG20k(AL)-β-Ala-Tyr-Nal(1)-Leu-Phe-Arg-Pro-Arg-Asn-NH₂ or a salt thereof;
[1B] PEG20k(AL)-β-Ala-Tyr-Nal(2)-Leu-Phe-Arg-Pro-Arg-Asn-NH₂ or a salt thereof;
[1C] PEG20k(AL)-NpipAc-Tyr-Nal(2)-Leu-Phe-Arg-Pro-Arg-Asn-NH₂ or a salt thereof;
[1D] PEG20k(AL)-NpipAc-Tyr-Nal(2)-Leu-Phe-Arg-Ala-Arg-Asn-NH₂ or a salt thereof;
[1E] PEG20k(AL)-PEG(2)-Tyr-Nal(2)-Leu-Phe-Arg-NMeAla-Arg-Asn-NH₂ or a salt thereof;
[1F] PEG20k(AL)-Pic(4)-Tyr-Nal(2)-Leu-Phe-Arg-NMeAla-Arg-Asn-NH₂ or a salt thereof;
[1G] PEG20k(AL)-Acp-Tyr-Nal(2)-Leu-Phe-Arg-NMeAla-Arg-Asn-NH₂ or a salt thereof;
[1H] PEG20k(AL)-β-Ala-Tyr-Nal(2)-Leu-Pya(4)-Arg-Pro-Arg-Asn-NH₂ or a salt thereof;
[1I] A prodrug of the peptide derivative or a salt thereof of item [1], [1A], [1B], [1C], [1D], [1E], [1F], [1G], or [1H];
[2] A medicament comprising the peptide derivative or a salt thereof, or a prodrug thereof, of item [1], [1A], [1B], [1C], [1D], [1E], [1F], [1G], or [1H];
[3] The medicament according to item [2], which is a neuromedin U receptor agonist;
[4] The medicament according to item [2], which is an anorectic agent;
[5] The medicament according to item [2], which is an agent for preventing or treating obesity;
[6] A method for preventing or treating obesity, comprising administering to a mammal an effective amount of the peptide derivative or a salt thereof, or a prodrug thereof, of item [1], [1A], [1B], [1C], [1D], [1E], [1F], [1G], or [1H]
[7] Use of the peptide derivative or a salt thereof, or a prodrug thereof, of item [1], [1A], [1B], [1C], [1D], [1E], [1F], [1G], or [1H], for producing an agent for preventing or treating obesity.
[8] A method for activating a neuromedin U receptor in a mammal, comprising administering to the mammal an effective amount of the peptide derivative or a salt thereof, or a prodrug thereof, of item [1], [1A], [1B], [1C], [1D], [1E], [1E], [1G], or [1H];
[9] A method for attenuating food intake in a mammal, comprising administering to the mammal an effective amount of the peptide derivative or a salt thereof, or a prodrug thereof, of item [1], [1A], [1B], [1C], [1D], [1E], [1F], [1G], or [1H];
[10] Use of the peptide derivative or a salt thereof, or a prodrug thereof, of item [1], [1A], [1B], [1C], [1D], [1E], [1F], [1G], or [1H], for producing an anorectic agent;
[11] The peptide derivative or a salt thereof, or a prodrug thereof, of item [1], [1A], [1B], [1C], [1D], [1E], [1F], [1G], or [1H], for preventing or treating obesity;
[12] The peptide derivative or a salt thereof, or a prodrug thereof, of item [1], [1A], [1B], [1C], [1D], [1E], [1F], [1G], or [1H], for attenuating food intake.

The above compound (I) and prodrug thereof are sometimes collectively referred to as “the compound of the present invention.”

### Advantageous Effects of Invention

The compound of the present invention is highly stable, and can exhibit a high antiobesity effect, even when administered in a usual manner, for example, peripherally. Thus, the compound is useful as an agent for preventing or treating obesity.

Further, the compound of the invention is useful as an anorectic agent, since the compound is highly stable and can exhibit a high anorectic effect. The compound of the invention acts on an NMUR2 selectively.

### DETAILED DESCRIPTION OF THE INVENTION

In the present specification, the term “C₁₋₆ alkyl group” refers to methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, etc., unless otherwise specified.

Examples of the “C₃₋₁₀ cycloalkyl group” as used herein include cyclopropyl, cyclobutyl, cyclopentyl, cyclohexyl, cycloheptyl, and cyclooctyl.

Examples of the “C₇₋₁₄ aralkyl group” as used herein include phenyl-C₁₋₂ alkyl groups such as benzyl, phenethyl, and benzhydryl; and α-naphthyl-C₁₋₂ alkyl groups such as α-naphthylmethyl.

Methyl (CH₃) may be hereinafter indicated as “Me” in accordance with commonly used abbreviations.

The abbreviations used herein to indicate amino acids, etc. are according to abbreviations defined in the IUPAC-IUB Commission on Biochemical Nomenclature or common abbreviations used in this field, examples of which are shown below.

For amino acids that may exist as optical isomers, their L-forms are denoted unless otherwise specified.

The abbreviations used are explained below.

Abbreviation: name or structural formula
Acp: 6-aminocaproic acid
Ala: alanine
Arg: arginine
Arg(Pbf): N^{ω}-2,2,4,6,7-pentamethyldihydrobenzofuransulfonyl-arginine
Asn: asparagine
Asn(Trt): N^{ω}-tritylasparagine
Asp: aspartic acid
Cys: cysteine
DIPCDI: 1,3-diisopropylcarbodiimide

### DMF: N,N-dimethylformamide

EDT: 1,2-ethanedithiol
Fmoc: 9-fluorenylmethoxycarbonyl
Gln: glutamine
Glu: glutamic acid
Gly: glycine
His: histidine
HOAt: 1-hydroxy-7-azabenzotriazole
Ile: isoleucine
Leu: leucine
Lys: lysine
Met: methionine
Nal(1): 1-naphthylalanine
Nal(2): 2-naphthylalanine
NMeAla: N^{α}-methylalanine
NpipAc: piperazin-1-ylacetyl

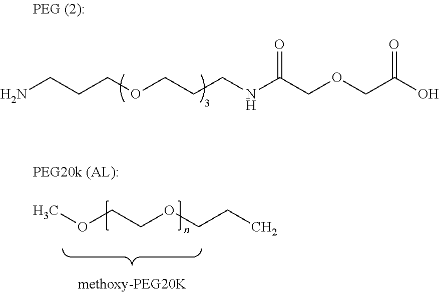

(wherein the portion represented by the formula

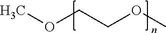

represents methoxy-PEG20k;
n is the number of repeating structural units, and is specified by a PEG20k molecular weight in the range of 16000 to 24000 (preferably 20000)).
Phe: phenylalanine
Pic (4): piperidin-4-carboxylic acid
Pro: praline
Pya(4): 4-pyridylalanine
Ser: serine
tBu: tert-butyl
TFA: trifluoroacetic acid
TIS: triisopropylsilane
Thr: threonine
Trp: tryptophan
Trt: trityl
Tyr: tyrosine

### Tyr(tBu): O-tert-butyltyrosine

Val: valine
β-Ala: β-alanine

In the specification, the peptides are shown in accordance with the conventional way of describing peptides; that is, the N-terminus (amino terminus) is shown on the left-hand side, And the C-terminus (carboxyl terminus) on the right-hand side.

The compound of the present invention is a peptide derivative selected from the group consisting of

PEG20k(AL)-β-Ala-Tyr-Nal(1)-Leu-Phe-Arg-Pro-Arg-Asn-NH₂,
PEG20k(AL)-β-Ala-Tyr-Nal(2)-Leu-Phe-Arg-Pro-Arg-Asn-NH₂,
PEG20k(AL)-NpipAc-Tyr-Nal(2)-Leu-Phe-Arg-Pro-Arg-Asn-NH₂,
PEG20k(AL)-NpipAc-Tyr-Nal(2)-Leu-Phe-Arg-Ala-Arg-Asn-NH₂,
PEG20k(AL)-PEG(2)-Tyr-Nal(2)-Leu-Phe-Arg-NMeAla-Arg-Asn-NH₂,
PEG20k(AL)-Pic(4)-Tyr-Nal(2)-Leu-Phe-Arg-NMeAla-Arg-Asn-NH₂,
PEG20k(AL)-Acp-Tyr-Nal(2)-Leu-Phe-Arg-NMeAla-Arg-Asn-NH₂, and
PEG20k(AL)-β-Ala-Tyr-Nal(2)-Leu-Pya(4)-Arg-Pro-Arg-Asn-NH₂; or a
salt thereof.

The compound of the present invention may be a salt. Examples of such salts include salts with inorganic bases, salts with organic bases, salts with inorganic acids, salts with organic acids, and salts with basic or acidic amino acids.

Preferable examples of salts with inorganic bases include alkali metal salts such as sodium salts and potassium salts; alkali earth metal salts such as calcium salts and magnesium salts; and aluminum salts and ammonium salts.

Preferable examples of salts with organic bases include salts with trimethylamine, triethylamine, pyridine, picoline, ethanolamine, diethanolamine, triethanolamine, dicyclohexylamine, N,N-dibenzylethylenediamine, or the like.

Preferable examples of salts with inorganic bases include salts with hydrochloric acid, hydrobromic acid, nitric acid, sulfuric acid, phosphoric acid, or the like.

Preferable examples of salts with organic acids include salts with formic acid, acetic acid, trifluoroacetic acid, fumaric acid, oxalic acid, tartaric acid, maleic acid, succinic acid, malic acid, methanesulfonic acid, benzenesulfonic acid, p-toluenesulfonic acid, or the like.

Preferable examples of salts with basic amino acids include salts with arginine, lysine, ornithine, or the like.

Preferable examples of salts with acidic amino acids include salts with aspartic acid, glutamic acid, or the like.

The amino acid sequence of 8 residues at the C-terminus of NMU is represented by SEQ ID NO.: 1 (Tyr-Phe-Leu-Phe-Arg-Pro-Arg-Asn-NH₂).

In the present specification, the “polypeptide consisting of an amino acid sequence set forth in SEQ ID NO.: 1 whose 1 or more amino acids are substituted”, which forms part of the compound of the present invention, may be simply referred to as “peptide (I)”. The first amino acid residue at the N-terminus is designated as the first position in accordance with the conventional way of describing peptides.

Peptide (I) used in the present invention is bound to a linker preferably at the α-amino group of the N-terminus.

These activities can be measured according to the methods described in the specification, or other known methods.

Peptide (I) is a polypeptide consisting of an amino acid sequence selected from SEQ ID NOs: 2 to 6:

SEQ ID NO: 2)
Tyr-Nal(1)-Leu-Phe-Arg-Pro-Arg-Asn-NH₂;
(SEQ ID NO: 3)
Tyr-Nal(2)-Leu-Phe-Arg-Pro-Arg-Asn-NH₂;
(SEQ ID NO: 4)
Tyr-Nal(2)-Leu-Phe-Arg-Ala-Arg-Asn-NH₂;
(SEQ ID NO: 5)
Tyr-Nal(2)-Leu-Phe-Arg-NMeAla-Arg-Asn-NH₂;
and
(SEQ ID NO: 6)
Tyr-Nal(2)-Leu-Pya(4)-Arg-Pro-Arg-Asn-NH₂.

As is clear from the above, the C-terminus in SEQ ID NOs: 2 to 6 is amidated (that is, —OH in the carboxyl group (—COOH) is replaced by NH₂).

Peptide (I) may be derived from the cells of warm-blooded animals (e.g., humans, mice, rats, guinea pigs, hamsters, rabbits, sheep, goats, swine, bovine, horses, birds, cats, dogs, monkeys, and chimpanzees) [e.g., splenocytes, nerve cells, glial cells, pancreatic β-cells, bone marrow cells, mesangial cells, Langerhans' cells, epidermal cells, epithelial cells, goblet cells, endothelial cells, smooth muscle cells, fibroblasts, fibrocytes, muscle cells, fat cells, immune cells (e.g., macrophages, T cells, B cells, natural killer cells, mast cells, neutrophils, basophils, eosinophils, monocytes, and dendritic cells), megakaryocytes, synovial cells, chondrocytes, osteocytes, osteoblasts, osteoclasts, mammary cells, hepatic cells or interstitial cells, and the corresponding precursor cells, stem cells, and cancer cells], or from any tissues where such cells are present [for example, brain or parts of the brain (e.g., olfactory bulb, amygdaloid nucleus, basal ganglia, hippocampus, thalamus, hypothalamus, cerebral cortex, medulla oblongata, and cerebellum), spinal cord, pituitary gland, stomach, pancreas, kidney, liver, gonads, thyroid gland, gall bladder, bone marrow, adrenal gland, skin, muscle, lung, gastrointestinal tract (e.g., large intestine and small intestine), blood vessel, heart, thymus, spleen, submandibular gland, peripheral blood, prostate, testis, ovary, placenta, uterus, bone, joint, adipose tissue, skeletal muscle, and peritoneum]. Peptide (I) may be synthesized chemically or in a cell-free translation system. Alternatively, the peptide (I) may be a genetically modified peptide produced from a transformant to which a nucleic acid containing a base sequence that encodes the amino acid sequence is induced.

Examples of the linker that forms part of the compound of the present invention include β-Ala, NpipAc, PEG (2), Pic(4), and Acp.

PEG20k(AL), which forms a part of the compound of the present invention, is as explained above. As explained above, PEG20k(AL) is bound to peptide (I) via a linker as mentioned above.

### [Production Method]

The method for producing the compound of the present invention will be explained below.

The compound of the present invention can be produced by binding PEG20k(AL) via a linker to peptide (I).

Peptide (I) can be prepared from the aforementioned warm-blooded animal cells or tissues by a known peptide purification method. Specifically, the tissues or cells of warm-blooded animals are homogenized, and the soluble fractions are isolated and purified by chromatography, such as reversed phase chromatography, ion exchange chromatography, and affinity chromatography, to prepare Peptide (I).

Peptide (I) can be produced according to a peptide synthesis method known per se.

The peptide synthesis method may be, for example, a solid phase synthesis method or a liquid phase synthesis method. A desired protein can be produced by condensing a partial peptide or amino acids that can form the compound of the present invention, and the remaining portion, and eliminating any protecting group the resultant product may have.

The condensation and elimination of the protecting group can be performed according to methods known per se, such as those described in (1) to (5) below:

### (1) M. Bodanszky and M. A. Ondetti, Peptide Synthesis, Interscience Publishers, New York (1966);

### (2) Schroeder and Luebke, The Peptide, Academic Press, New York (1965);

(3) Nobuo Izumiya, et al.: Peptide Gosei-no-Kiso to Jikken (Peptide Synthesis Fundamentals and Experiments), published by Maruzen Co. (1975);

### (4) Haruaki Yajima and Shunpei Sakakibara: Seikagaku Jikken Koza (Biochemistry Experiment Lecture Series) 1, Tanpakushitsu no Kagaku (Protein Chemistry) IV, 205 (1977); and

(5) Haruaki Yajima, ed.: Zoku Iyakuhin no Kaihatsu (Second Series Drug Development), Vol. 14, Peptide Synthesis, published by Hirokawa Shoten.

The compound of the present invention thus obtained can be isolated and purified by known purification methods.

Further, peptide (I) can also be produced by culturing a transformant containing a nucleic acid that encodes the peptide, and isolating and purifying peptide (I) from the obtained culture.

The nucleic acid that encodes peptide (I) may be DNA or RNA, or a DNA/RNA chimera; and is preferably DNA. The nucleic acid may be double-stranded or single-stranded. The double-stranded nucleic acid may be double-stranded DNA, double-stranded RNA, or a DNA-RNA hybrid. The single-stranded nucleic acid may be a sense strand (i.e., coding strand) or an antisense strand (i.e., non-coding strand).

Examples of DNA that encodes peptide (I) include genomic DNA; cDNA derived from any cells of warm-blooded animals (e.g., humans, mice, rats, guinea pigs, hamsters, rabbits, sheep, goats, swine, bovine, horses, birds, cats, dogs, monkeys, and chimpanzees) [e.g., splenocytes, nerve cells, glial cells, pancreatic β-cells, bone marrow cells, mesangial cells, Langerhans' cells, epidermal cells, epithelial cells, endothelial cells, fibroblasts, fibrocytes, muscle cells, fat cells, immune cells (e.g., macrophages, T cells, B cells, natural killer cells, mast cells, neutrophils, basophils, eosinophils, monocytes, and dendritic cells), megakaryocytes, synovial cells, chondrocytes, osteocytes, osteoblasts, osteoclasts, mammary cells, hepatic cells or interstitial cells, and the corresponding precursor cells, stem cells, or cancer cells, and blood cells] or from any tissues where such cells are present [for example, brain or parts of the brain (e.g., olfactory bulb, amygdaloid nucleus, basal ganglia, hippocampus, thalamus, hypothalamus, subthalamic nucleus, cerebral cortex, medulla oblongata, cerebellum, occipital lobe, frontal lobe, temporal lobe, putamen, caudate nucleus, corpus callosum, nigra), spinal cord, pituitary gland, stomach, pancreas, kidney, liver, gonad, thyroid gland, gall bladder, bone marrow, adrenal gland, skin, muscle, lung, gastrointestinal tract (e.g., large intestine and small intestine), blood vessel, heart, thymus, spleen, submandibular gland, peripheral blood, peripheral hemocyte, prostate, testis, ovary, placenta, uterus, bone, joint, skeletal muscle, and peritoneum]; and synthetic DNA.

The genomic DNA and cDNA that encode peptide (I) can be directly amplified according to a method known per se, for example, the Polymerase Chain Reaction (hereinafter abbreviated as the “PCR”) and the Reserve Transcriptase-PCR (hereinafter referred to as the “RT-PCR”) using a genomic DNA fraction and total RNA or a mRNA fraction prepared from the aforementioned cells or tissues as templates. Alternatively, the genomic DNA and cDNA that encode peptide (I) can be respectively cloned from a genomic DNA library and a cDNA library that are prepared by inserting genomic DNA and total RNA or a mRNA fragment prepared from the aforementioned cells and tissues into an appropriate vector, by a method known per se, such as colony or plaque hybridization or PCR. The vector to be used in the libraries may be, for example, any of bacteriophages, plasmids, cosmids, and phagemids.

The compound of the present invention can be synthesized, for example, by any of the following methods.

(1) A PEGylation reagent containing an aldehyde (e.g., SUNBRIGHT ME-300-AL (trade name), NOF Corporation) is bound to the amino group of peptide (I).
(2) ω-aminocarboxylic acid or α-amino acid is introduced as a linker to the N-terminal amino group of peptide (I), and a PEGylation reagent containing an aldehyde group (e.g., SUNBRIGHT ME-300AL (trade name), NOF Corporation) is reacted with the amino group derived from this linker. In this case, the linker in the compound of the present invention is derived from the PEGylation reagent and ω-aminocarboxylic acid, or the PEGylation reagent and α-amino acid.

The aforementioned reagents can be obtained, for example, as commercial products. Each reaction can be carried out by a method known to those in the art.

Examples of compounds that can be preferably used as an intermediate in the production of the compound of the invention include compounds comprising: a polypeptide consisting of an amino acid sequence set forth in one of SEQ ID NOs.: 2 to 6; and a linker that is bound to the N-terminus of the polypeptide.

Specific examples of such intermediates include the following:

### β-Ala-Tyr-Nal(1)-Leu-Phe-Arg-Pro-Arg-Asn-NH₂,

### β-Ala-Tyr-Nal(2)-Leu-Phe-Arg-Pro-Arg-Asn-NH₂,

### NpipAc-Tyr-Nal(2)-Leu-Phe-Arg-Pro-Arg-Asn-NH₂,

### NpipAc-Tyr-Nal(2)-Leu-Phe-Arg-Ala-Arg-Asn-NH₂,

### PEG(2)-Tyr-Nal(2)-Leu-Phe-Arg-NMeAla-Arg-Asn-NH₂.

### Pic(4)-Tyr-Nal(2)-Leu-Phe-Arg-NMeAla-Arg-Asn-NH₂,

### Acp-Tyr-Nal(2)-Leu-Phe-Arg-NMeAla-Arg-Asn-NH₂, and

### β-Ala-Tyr-Nal(2)-Leu-Pya(4)-Arg-Pro-Arg-Asn-NH₂.

The above intermediates may be used in the farm of salts. Examples of such salts include those mentioned above as examples of salts of the compound of the present invention.

Examples of the protecting groups used to protect the amino group in the starting amino acid include Z, Boc, tert-pentyloxycarbonyl, isobornyloxycarbonyl, 4-methoxybenzyloxycarbonyl, Cl—Z, adamantyloxycarbonyl, trifluoroacetyl, phthaloyl, formyl, 2-nitrophenylsulphenyl, diphenylphosphinothioyl, Fmoc, and trityl.

Examples of the protecting groups used to protect the carboxyl group in the starting amino acid include C₁₋₆ alkyl, C₃₋₁₀ cycloalkyl, C₇₋₁₄ aralkyl, allyl, 2-adamantyl, 4-nitrobenzyl, 4-methoxybenzyl, 4-chlorobenzyl, phenacyl, benzyloxycarbonyl hydrazide, tert-butoxycarbonyl hydrazide, and tritylhydrazide.

The hydroxyl group of serine and threonine can be protected by, for example, esterification or etherification.

Examples of groups suitably used for the esterification include lower (C₂₋₄) alkanoyl groups such as acetyl; aroyl groups such as benzoyl; and groups derived from organic acids. Examples of groups suitably used for the etherification include benzyl, tetrahydropyranyl, tert-butyl (Bu^{t}), and trytyl (Trt).

Examples of protecting groups for the phenolic hydroxyl group of tyrosine include Bzl, 2,6-dichlorobenzyl, 2-nitrobenzyl, Br—Z, and tert-butyl.

Examples of protecting groups for the imidazole moiety of histidine include Tos, 4-methoxy-2,3,6-trimethylbenzenesulfonyl (Mtr), DNP, Bom, Bum, Boc, Trt, and Fmoc.

Examples of protecting groups for the guanidine group of arginine include Tos, Z, 4-methoxy-2,3,6-trimethylbenzenesulfonyl (Mtr), p-methoxybenzenesulfonyl (MBS), 2,2,5,7,8-pentamethylchroman-6-sulfonyl (Pmc), mesitylene-2-sulfonyl (Mts), 2,2,4,6,7-pentamethyldihydrobenzofuran-5-sulfonyl (Pbf), Boc, Z, and NO₂.

Examples of protecting groups for the side chain amino group of lysine include Z, Cl—Z, trifluoroacetyl, Boc, Fmoc, Trt, Mtr, and 4,4-dimethyl-2,6-dioxocyclohexylideneyl (Dde).

Examples of protecting groups for the indolyl of tryptophan include formyl (For), Z, Boc, Mts, and Mtr.

Examples of protecting groups for asparagine and glutamine include Trt, xanthyl (Xan), 4,4′-dimethoxybenzhydryl (Mbh), and 2,4,6-trimethoxybenzyl (Tmob).

Examples of activated carboxyl groups in the starting material include the corresponding acid anhydrides, azides, and activated esters [esters with alcohols (e.g., pentachlorophenol, 2,4,5-trichlorophenol, 2,4-dinitrophenol, cyanomethyl alcohol, p-nitrophenol, HONB, N-hydroxysuccimide, 1-hydroxybenzotriazole (HOBt), and 1-hydroxy-7-azabenzotriazole (HOAt)]. Examples of activated amino groups in the starting material include the corresponding phosphorous amides.

Methods of removing (eliminating) the protecting groups include catalytic reduction under hydrogen gas flow in the presence of a catalyst such as Pd-black or Pd-carbon; an acid treatment with anhydrous hydrogen fluoride, methanesulfonic acid, trifluoromethanesulfonic acid, trifluoroacetic acid, trimethylsilyl bromide (TMSBr), trimethylsilyl trifluoromethanesulfonate, tetrafluoroboric acid, tris(trifluoro)boron, boron tribromide, or a mixed solution thereof; a base treatment with diisopropylethylamine, triethylamine, piperidine, piperazine, etc.; and reduction with sodium in liquid ammonia. The elimination of protecting groups by the acid treatment described above is typically carried out at a temperature of −20° C. to 40° C. In the acid treatment, it is efficient to add a cation scavenger such as anisole, phenol, thioanisole, m-cresol, or p-cresol; dimethylsulfide, 1,4-butanedithiol, 1,2-ethanedithiol, etc. Furthermore, the 2,4-dinitrophenyl group used as a protecting group for the imidazole moiety of histidine is removed by a treatment with thiophenol. The formyl group used as a protecting group for the indole of tryptophan is removed by the above-mentioned acid treatment in the presence of 1,2-ethanedithiol, 1,4-butanedithiol, etc., as well as by a treatment with an alkali such as a dilute sodium hydroxide solution or dilute ammonia.

Protection of functional groups that should not be involved in the reaction of the starting material, the kind of protecting group to be used, elimination of the protecting group, and activation of functional groups that are involved in the reaction may be appropriately selected from known protecting groups and known means.

When the compound of the present invention is obtained in a free state by the aforementioned synthetic method, it may be converted to a salt according to a usual method. When the compound of the present invention is obtained as a salt, it can be converted to a free form or other salts according to a usual method. The compound of the present invention thus obtained can be isolated and purified from the reaction solution by a known means, such as phase transfer, concentration, solvent extraction, fractional distillation, crystallization, recrystallization, and chromatography.

When the compound of the present invention is present in the form of a configurational isomer, diastereomer, conformer, etc., each can be isolated by the above-mentioned separation and purification means, if desired. When the compound is racemic, it can be separated into an S-form and an R-form by usual optical resolution means.

When the compound of the present invention is present in the form of a stereoisomer, each of the individual isomers and a mixture thereof are included within the scope of the present invention.

The compound of the present invention may be a hydrate or non-hydrate. Further, the compound of the present invention may be a solvate or a non-solvate.

The compound of the present invention may be labeled with an isomer (e.g., ³H, ¹⁴C, ¹⁸F, ³⁵S, or ¹²⁵I), etc. Further, the compound of the present invention may be substituted with deuterium.

The compound of the present invention is useful as an agent for preventing or treating obesity, or as an anorectic agent.

The compound of the present invention, which has high safety and low toxicity, and causes fewer adverse effects such as vomiting, diarrhea, etc, can be administered as an agent for preventing or treating obesity or an anorectic agent to mammals (e.g., humans, mice, rats, rabbits, sheep, swine, bovine, horses, birds, cats, dogs, monkeys, and chimpanzees), for example, peripherally.

The compound of the present invention can be used as an agent for preventing or treating symptomatic obesity, simple obesity, a disease or condition associated with obesity, eating disorders, etc.

Examples of symptomatic obesity include endocrine obesity (e.g., Cushing's syndrome, hypothyroidism, insulinoma, obese type II diabetes, pseudohypoparathyroidism, and hypogonadism); central obesity (e.g., hypothalamic obesity, frontal lobe syndrome, Kleine-Levin syndrome); hereditary obesity (such as Prader-Willi syndrome and Laurence-Moon-Biedl syndrome); and drug-induced obesity (e.g., obesity due to steroid, phenothiazine, insulin, sulfonylurea (SU) agent, and β-blocker).

Examples of a disease or condition associated with obesity include impaired glucose tolerance, diabetes (in particular, type 2 diabetes and obese diabetes), dyslipidemia (e.g., hypercholesterolemia, hyper-LDL-cholesterolemia, hypo-HDL-cholesterolemia, postprandial hyperlipidemia, and hypertriglyceridemia), hypertension, heart failure, hyperuricemia/gout, fatty liver (including non-alcoholic steato-hepatitis), coronary artery disease (e.g., myocardial infarction, angina pectoris), cerebral infarction (e.g., cerebral thrombosis, and transient ischemic attack), bone and joint disease (e.g., gonarthrosis, coxarthrosis, spondylitis deformans, and lower back pain), sleep apnea syndrome/Pickwick syndrome, menstrual disorders (e.g., abnormal menstrual cycles, menstrual flow and cycle disorders, amenorrhea, abnormal menstruation-related symptoms, metabolic syndrome [pathology having three or more diseases or conditions selected from hypertriglyceridemia (TG), hypo-HDL-cholesterolemia (HDL-C), hypertension, abdominal obesity, and inadequate glucose tolerance).

The compound of the present invention can also be used for the secondary prophylaxis or inhibition of the progression of the above-mentioned various diseases (e.g., cardiovascular events such as myocardial infarction).

The compound of the present invention is useful as an anorectic agent or as a body weight-gain inhibitor.

The compound of the present invention can be concurrently used with diet therapy (e.g., diet therapy for diabetes) and/or exercise therapy.

The compound of the present invention can also be used as an agent for preventing or treating borderline diabetes, inadequate glucose tolerance, IFG (Impaired Fasting Glucose), and IFG (Impaired Fasting Glycemia). Further, the compound of the present invention can prevent the progression of borderline diabetes, inadequate glucose tolerance, IFG (Impaired Fasting Glucose), and IFG (Impaired Fasting Glycemia) into diabetes.

In addition, the compound of the present invention can also be used as an agent for preventing or treating diabetic complications [e.g., neuropathy, nephropathy, retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infection, and inferior limb infection), diabetic gangrene, xerostomia, hypacusis, cerebrovascular disorder, and peripheral blood circulation disorder].

The compound of the present invention is typically used as a pharmaceutical composition obtained by formulating the compound with a pharmacologically acceptable carrier according to a known method (e.g., a method described in the Japanese Pharmacopoeia).

As pharmacologically acceptable carriers, various organic or inorganic carrier substances conventionally used as materials for pharmaceutical preparations can be used. Examples of such carriers include excipients, lubricants, binders, and disintegrants for solid preparations; and solvents, solubilizers, suspending agents, isotonizing agents, buffers, and soothing agents for liquid preparations. If necessary, additives for pharmaceutical preparations, such as preservatives, antioxidants, colorants, and sweeteners, may be used to formulate such preparations.

Preferable examples of excipients include lactose, sucrose, D-mannitol, D-sorbitol, starch, gelatinized starch, dextrin, crystalline cellulose, low-substituted hydroxypropyl cellulose, sodium carboxymethylcellulose, gum arabic, pullulan, light anhydrous silicic acid, synthetic aluminum silicate, magnesium aluminometasilicate, xylitol, sorbitol, and erythritol.

Preferable examples of lubricants include magnesium stearate, calcium stearate, talc, colloidal silica, and polyethylene glycol 6000.

Preferable examples of binders include gelatinized starch, sucrose, gelatin, gum arabic, methylcellulose, carboxymethylcellulose, sodium carboxymethylcellulose, crystalline cellulose, sucrose, D-mannitol, trehalose, dextrin, pullulan, hydroxypropyl cellulose, hydroxypropyl methylcellulose, and polyvinylpyrrolidone.

Preferable examples of disintegrants include lactose, sucrose, starch, carboxymethylcellulose, calcium carboxymethylcellulose, croscarmellose sodium, sodium carboxymethyl starch, low-substituted hydroxypropylcellulose, light anhydrous silicic acid, and calcium carbonate.

Preferable examples of solvents include water for injection, saline, Ringer's solution, alcohol, propylene glycol, polyethylene glycol, sesame oil, corn oil, olive oil, and cottonseed oil.

Preferable examples of solubilizers include polyethylene glycol, propylene glycol, D-mannitol, trehalose, benzylbenzoate, ethanol, tris-aminomethane, cholesterol, triethanolamine, sodium carbonate, sodium citrate, sodium salicylate, and sodium acetate.

Preferable examples of suspending agents include surfactants such as stearyltriethanolamine, sodium lauryl sulfate, lauryl aminopropionic acid, lecithin, benzalkonium chloride, benzethonium chloride, and glycerol monostearate; hydrophilic polymers such as polyvinyl alcohol, polyvinylpyrrolidone, sodium carboxymethylcellulose, methylcellulose, hydroxymethylcellulose, hydroxyethylcellulose, and hydroxypropylcellulose; polysorbates and polyoxyethylene hydrogenated castor oil.

Preferable examples of isotonizing agents include sodium chloride, glycerin, D-mannitol, D-sorbitol, glucose, xylitol, and fructose.

Preferable examples of buffers include buffer solutions such as phosphates, acetates, carbonates, and citrates.

Preferable examples of soothing agents include propylene glycol, lidocaine hydrochloride, and benzyl alcohol.

Preferable examples of preservatives include para-oxybenzoic acid esters, chlorobutanol, benzyl alcohol, phenethyl alcohol, dehydroacetic acid, and sorbic acid.

Preferable examples of antioxidants include sulfites and ascorbates.

Preferable examples of colorants include water-soluble edible tar pigments (e.g., food colors such as Food Color Red Nos. 2 and 3, Food Color Yellow Nos. 4 and 5, and Food Color Blue Nos. 1 and 2), water-insoluble lake pigments (e.g., aluminum salts of the aforementioned water-soluble edible tar pigments), and natural pigments (e.g., 3-carotene, chlorophil, and red iron oxide).

Preferable examples of sweeteners include sodium saccharin, dipotassium glycyrrhizate, aspartame, and stevia.

The medicament containing the compound of the present invention can be formulated, alone or with a pharmaceutically acceptable carrier, into tablets (including sugar-coated tablets, film-coated tablets, sublingual tablets, orally disintegrable tablets, and buccal tablets), pills, powders, granules, capsules (including soft capsules and microcapsules), troches, syrups, liquids, emulsions, suspensions, controlled-release formulations (e.g., quick-release formulations, sustained release formulations, and sustained release microcapsules), aerosols, films (e.g., orally disintegrable films, and film for application to oral mucosa), injections (e.g., subcutaneous injections, intravenous injections, intramuscular injections, and intraperitoneal injections), intravenous drips, transdermal preparations, ointments, lotions, patches, suppositories (e.g., rectal suppositories and vaginal suppositories), pellets, transnasal agents, pulmonary preparations (inhalants), eye drops, etc., according to methods known per se (e.g., methods described in the Japanese Pharmacopoeia), and safely administered orally or parenterally (for example, via an intravenous, intramuscular, subcutaneous, intraorgan, intranasal, intracutaneous, instillation, intracerebral, intrarectal, intravaginal, interperitoneal, or intratumoral route; in the vicinity of the tumor; or directly to the lesion).

The content of the compound of the present invention in the pharmaceutical compositions is, for example, 0.1 to 100 wt. %.

The methods for producing oral preparations (e.g., tablets, pills, powders, granules, capsules, troches, syrups, liquids, emulsions, suspensions, controlled-release formulations, aerosols, and films) and parenteral preparations (e.g., injections, intravenous drips, transdermal preparations, ointments, lotions, patches, suppositories, pellets, transnasal agents, pulmonary preparations, and eye drops) are specifically explained below. Oral preparations can be produced by adding, for example, an excipient (e.g., lactose, sucrose, starch, D-mannitol, xylitol, sorbitol, erythritol, crystalline cellulose, and light anhydrous silicic acid), a disintegrant (e.g., calcium carbonate, starch, carboxymethylcellulose, calcium carboxymethylcellulose, low-substituted hydroxypropylcellulose, croscarmellose sodium, sodium carboxymethyl starch, and light anhydrous silicic acid), a binder (e.g., gelatinized starch, gum arable, carboxymethylcellulose, hydroxypropylcellulose, hydroxypropylmethylcellulose, polyvinylpyrrolidone, crystalline cellulose, methylcellulose, sucrose, D-mannitol, trehalose, and dextrin), a lubricant (e.g., talc, magnesium stearate, calcium stearate, colloidal silica, and polyethylene glycol 6000), etc. to the active ingredient, and compression-molding the mixture.

Further, oral preparations may be coated by a method known per se for the purpose of masking of the taste, enteric coating, or sustained release. Examples of usable coating agents include enteric polymers (e.g., cellulose acetate phthalate, methacrylic acid copolymer L, methacrylic acid copolymer LD, methacrylic acid copolymer S, hydroxypropylmethylcellulose phthalate, hydroxypropylmethylcellulose acetate succinate, and carboxymethylethylcellulose), gastrosoluble polymers (e.g., polyvinylacetal diethylaminoacetate, and aminoalkyl methacrylate copolymer E), water-soluble polymers (e.g., hydroxypropylcellulose, and hydroxypropylmethylcellulose), water insoluble polymers (e.g., ethyl cellulose, aminoalkyl methacrylate copolymer RS, and ethyl acrylate-methyl methacrylate copolymer), and waxes. For coating, plasticizers such as polyethylene glycol, and light-shielding agents such as titanium oxide and iron sesquioxide may be used together with the above-mentioned coating agents.

Injections can be produced by dissolving, suspending, or emulsifying the active ingredient in an aqueous solvent (e.g., distilled water, saline, and Ringer's solution) or an oily solvent (e.g., a vegetable oil such as olive oil, sesame oil, cottonseed oil, and corn oil; propylene glycol, macrogol, and tricaprylin) together with a dispersing agent (e.g., Tween 80 (manufactured by Atlas Powder, USA), HCO 60 (manufactured by Nikko Chemicals Co., Ltd.), polyethyleneglycol, carboxymethylcellulose, and sodium alginate), a preservative (e.g., methylparaben, propylparaben, benzyl alcohol, chlorobutanol, and phenol), an isotonizing agent (e.g., sodium chloride, glycerine, D-sorbitol, D-mannitol, xylitol, glucose, and fructose). In this case, if desired, the following additives may be added: a solubilizer (e.g., sodium salicylate, sodium acetate, polyethylene glycol, propylene glycol, D-mannitol, trehalose, benzyl benzoate, ethanol, tris-aminomethane, cholesterol, triethanolamine, sodium carbonate, and sodium citrate), a suspending agent (e.g., surfactants such as stearyl triethanolamine, sodium laurylsulfate, lauryl aminopropionic acid, lecithin, benzalkonium chloride, benzethonium chloride, and glycerol monostearate; and hydrophilic polymers such as polyvinyl alcohol, polyvinyl pyrrolidone, sodium carboxymethylcellulose, methylcellulose, hydroxymethylcellulose, hydroxyethylcellulose and hydroxypropylcellulose), a buffer (e.g., buffer solutions such as phosphates, acetates, carboxylates, and citrates), a stabilizer (e.g., human serum albumin), a soothing agent (e.g., propylene glycol, lidocaine hydrochloride, and benzyl alcohol), and a preservative (e.g., p-oxybenzoic acid esters, chlorobutanol, benzalkonium chloride, benzyl alcohol, phenethyl alcohol, dehydroacetic acid, and sorbic acid).

External preparations (e.g., transdermal formulations, ointments, lotions, and patches) can be produced by formulating the active ingredient into solid, semi-solid, or liquid compositions.

For example, solid compositions as mentioned above can be produced by pulverizing the active ingredient as is, or by adding an excipient (e.g., lactose, D-mannitol, starch, crystalline cellulose, and sucrose), a thickener (e.g., natural gums, cellulose derivatives, and acrylic acid polymers) to the active ingredient, mixing them, and then pulverizing the mixture. Liquid compositions as mentioned above can be produced in almost the same manner as the injections. Semi-solid compositions are preferably in the form of an aqueous or oily gel, or an ointment. All of these compositions may also contain a pH modulating agent (e.g., phosphoric acid, citric acid, hydrochloric acid, and sodium hydroxide), or a preservative (e.g., p-oxybenzoic acid esters, chlorobutanol, benzalkonium chloride, benzylalcohol, phenethylalcohol, dehydroacetic acid, and sorbic acid). Suppositories can be produced by formulating the active ingredient into an oily or aqueous, solid, semi-solid, or liquid composition. Examples of oily bases usable in the production of the composition include higher fatty acid glycerides (e.g., cacao butter, and Witepsols), medium fatty acid triglycerides (e.g., Miglyols), and vegetable oils (e.g., sesame oil, soybean oil, and cottonseed oil). Examples of aqueous bases include polyethyleneglycols and propyleneglycol. Examples of aqueous gel bases include natural gums, cellulose derivatives, vinyl polymers, and acrylic acid polymers.

The dose of the compound of the present invention can be appropriately selected according to the administration subject, administration route, target disease, clinical symptoms, etc. For example, when the pharmaceutical composition containing the compound of the present invention as an active ingredient is subcutaneously administered to an adult, the compound of the present invention as an active ingredient is typically given in a single dose of about 5 to 100,000 μg per human, and preferably about 500 to 10,000 μg per human. This dose is preferably administered once to three times a day.

The compound of the present invention may be used concomitantly with other drugs having no adverse effects on the compound of the present invention for the purpose of enhancing the activity (e.g., an anorectic effect, and a preventive or therapeutic effect on obesity) of the compound of the invention or reducing the amount thereof. Examples of such drugs include “agents for treating diabetes”, “agents for treating diabetic complications”, “agents for treating obesity”, and “agents for treating hyperlipidemia”). Two or more such drugs (hereinafter sometimes simply referred to as “concomitant drugs”) may be combined at an appropriate ratio for use.

Examples of the “agents for treating diabetes” include insulin preparations (e.g., animal insulin preparations extracted from pancreas of bovine and swine; human insulin preparations genetically synthesized using Escherichia coli and yeast; zinc insulin; protamine zinc insulin; fragments or derivatives of insulin (e.g., INS-1), and oral insulin preparations), insulin sensitizers (e.g., pioglitazone or a salt thereof (preferably hydrochloride), rosiglitazone or a salt thereof (preferably maleate), Tesaglitazar, Ragaglitazar, Muraglitazar, Edaglitazone, Metaglidasen, Naveglitazar, AMG-131, THR-0921, α-glucosidase inhibitors (e.g., voglibose, acarbose, miglitol, and emiglitate), biguanides (e.g., metformin, buformin, and their salts (e.g., hydrochloride, fumarate, and succinate)), insulin secretagogues [sulfonylureas (e.g., tolbutamide, glibenclamide, gliclazide, chlorpropamide, tolazamide, acetohexamide, glyclopyramide, glimepiride, glipizide, and glybuzole), repaglinide, nateglinide, and mitiglinide or a calcium salt hydrate thereof), dipeptidyl-peptidase IV inhibitors (e.g., Vildagliptin, Sitagliptin, Saxagliptin, T-6666, and TS-021), 33 agonists (e.g., AJ-9677), GPR40 agonist, GLP-1 receptor agonists [e.g., GLP-1, GLP-1MR agent, NN-2211, AC-2993 (exendin-4), BIM-51077, Aib (8,35) hGLP-1 (7,37)NH₂, and CJC-1131], amylin agonists (e.g., pramlintide), phosphotyrosine phosphatase inhibitors (e.g., sodium vanadate), gluconeogenesis inhibitors (e.g., glycogen phosphorylase inhibitors, glucose-6-phosphatase inhibitors, and glucagon antagonists), SGLUT (sodium-glucose cotransporter) inhibitors (e.g., T-1095), 11β-hydroxysteroid dehydrogenase inhibitors (e.g., BVT-3498), adiponectin or adiponectin agonists, IKK inhibitors (e.g., AS-2868), leptin resistance-improving drugs, somatostatin receptor agonists, glucokinase activators (e.g., Ro-28-1675), and GIP (glucose-dependent insulinotropic peptide) receptor agonists.

Examples of the “agents for treating diabetic complications” include aldose reductase inhibitors (e.g., tolrestat, epalrestat, zenarestat, zopolrestat, minairestat, fidarestat, and ranirestat), neurotrophic factors and neurotrophic factor-increasing drugs (e.g., NGF, NT-3, BDNF, neurotrophic factor production-secretion promoters described in WO01/14372 (e.g., 4-(4-chlorophenyl)-2-(2-methyl-1-imidazolyl)-5-[3-(2-methylphenoxy)propyl]oxazole)), PKC inhibitors (e.g., ruboxistaurin mesylate), AGE inhibitors (e.g., ALT946, pimagedine, N-phenacylthiazolium bromide, EXO-226, pyridorin, and pyridoxamine), active oxygen scavengers (e.g., thioctic acid), cerebral vasodilators (e.g., tiapride and mexiletine), somatostatin receptor agonists (e.g., BIM23190), apoptosis signal regulating kinase-1 (ASK-1) inhibitors, and neuronal regeneration promoters (e.g., Y-128, VX-853, and prosaptide).

Examples of the “antiobesity agents” include central antiobesity agents (e.g., dexfenfluramine, fenfluramine, phentermine, sibutramine, amfepramone, dexamphetamine, mazindol, phenylpropanolamine, and clobenzorex; neuropeptide Y antagonists (e.g., CP-422935); cannabinoid receptor antagonists (e.g., SR-141716 and SR-147778); ghrelin antagonists; 11β-hydroxysteroid dehydrogenase inhibitors (e.g., BVT-3498), pancreatic lipase inhibitors (e.g., orlistat, cetilistat, β3 agonist (e.g., AJ-9677), peptide antifeedants (e.g., leptin, CNTF (Ciliary Neurotrophic Factor), cholecystokinin agonists (e.g., lintitript, and FPL-15849), and anorectic agents (e.g., P-57).

Examples Of the “agents for treating hyperlipidemia” include HMG-CoA reductase inhibitors (e.g., pravastatin, simvastatin, lovastatin, atorvastatin, fluvastatin, rosuvastatin, pitavastatin, and their salts (e.g., sodium salts and calcium salts)), squalene synthase inhibitors (e.g., the compounds described in WO 97/10224, for example, N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid), fibrate compounds (e.g., bezafibrate, clofibrate, simfibrate, and clinofibrate), ACAT inhibitors (e.g., avasimibe, and eflucimibe), anion exchange resins (e.g., colestyramine), probucol, nicotinic acid drugs (e.g., nicomol and niceritrol), ethyl icosapentate, and phytosterols (e.g., soysterol and γ-oryzanol).

The timing of administration of the concomitant drug is not limited. The compound of the present invention and the concomitant drug may be administered to the subject simultaneously, or separately at staggered intervals. The dosage of the concomitant drug may be determined based on the dose clinically used, and can be appropriately selected depending on the administration subject, administration route, disease, combination, etc.

The manner of administration of the concomitant drug with the compound of the present invention is not particularly limited, insofar as the compound of the present invention and the concomitant drugs are administered in combination. Examples of the manner of administration are as follows:

(1) administration of a single preparation obtained by simultaneously formulating the compound of the present invention with the concomitant drug;
(2) simultaneous administration of two kinds of preparations, which are obtained by separately formulating the compound of the present invention and the concomitant drug, by a single administration route;
(3) staggered-interval administration of two kinds of preparations, which are obtained by separately formulating the compound of the present invention and the concomitant drug, by the same administration route;
(4) simultaneous administration of two kinds of preparations, which are obtained by separately formulating the compound of the present invention and the concomitant drug, by different administration routes; and
(5) staggered-interval administration of two kinds of preparations, which are obtained by separately formulating the compound of the present invention and the concomitant drug, by different administration routes (for example, administration in the order of the compound of the present invention and the concomitant drug, or in the reverse order).

The mixing ratio of the compound of the present invention and the concomitant drug can be appropriately selected according to the administration subject, administration route, disease, etc.

### EXAMPLES

Hereinafter, the present invention is described with reference to Reference Examples, Examples, Test Examples and Formulation Examples. However, the present invention is not limited thereto.

In the present invention, Tyr-Phe-Leu-Phe-Arg-Pro-Arg-Asn-NH₂ (SEQ ID NO.: 1) is sometimes expressed as NMU-8.

The number shown after an amino acid represents the amino acid number. The amino acid numbers in SEQ ID NO.: 1 are shown below. Specifically, the position of Tyr at the N-terminus of NMU-8 is regarded as 1 and the position of Asn at the C-terminus is regarded as 8.

Tyr-Phe-Leu-Phe-Arg-Pro-Arg-Asn-NH₂
1 2 3 4 5 6 7 8

For example, β-Ala0,Nal(1)2-NMU-8, i.e., Compound 1 (Reference Example 1), represents a peptide in which β-Ala is extended to the N-terminus (position 0) of NMU-8, and Phe at position 2 is replaced by Nal(1).

Note that the above is a convenient notation; the β-Ala is a linker, and does not form the polypeptide used in the present invention.

The following are the compounds used in the Reference Examples, Examples, and Test Examples.

Each of the bonding hands “-” between XX0, XX1, XX2, XX3, XX4, XX5, XX6, XX7, XX8, and NH₂ in the formula “XX0-XX1-XX2-XX3-XX4-XX5-XX6-XX7-XX8-NH₂” represents the following.

The bonding hand “-” in the formula “XX0-XX1” represents a bond between a group represented by XX0 or the carboxyl group (carboxyl group at the α-position) in XX0 and the amino group (amino group at the α-position) in XX1. More specifically, the formula “XX0-XX1” indicates that the hydrogen atom of the amino group (NH₂) in XX1 is replaced by a group represented by XX0, or that the carboxyl group (—COOH) in XX0 and the amino group (NH₂) in XX1 form an amide bond.

The bonding hand “-” in the formula “XX1-XX2” indicates that the carboxyl group (carboxyl group at the α-position) in XX1 and the amino group (amino group at the α-position) in XX2 form an amide bond. The bonding hands “-” in the formulae “XX2-XX3,” “XX3-XX4,” “XX4-XX5,” “XX5-XX6,” “XX6-XX7,” and “XX7-XX8” have the same meaning as described above.

The bonding hand “-” in the formula “XX8-NH₂” represents a bond between the carboxyl group (carboxyl group at the α-position) in XX8 and —NH₂. More specifically, the formula “XX8-NH₂” indicates that —OH of the carboxyl group (—COOH) in XX8 is replaced by —NH₂.

In PEG-modified compounds of the above, the number and k attached to the “PEG” represent the molecular weight (kDa) of PEG; and (AL) represents —(CH₂)₃—.

The following are examples of the compounds of the present invention:

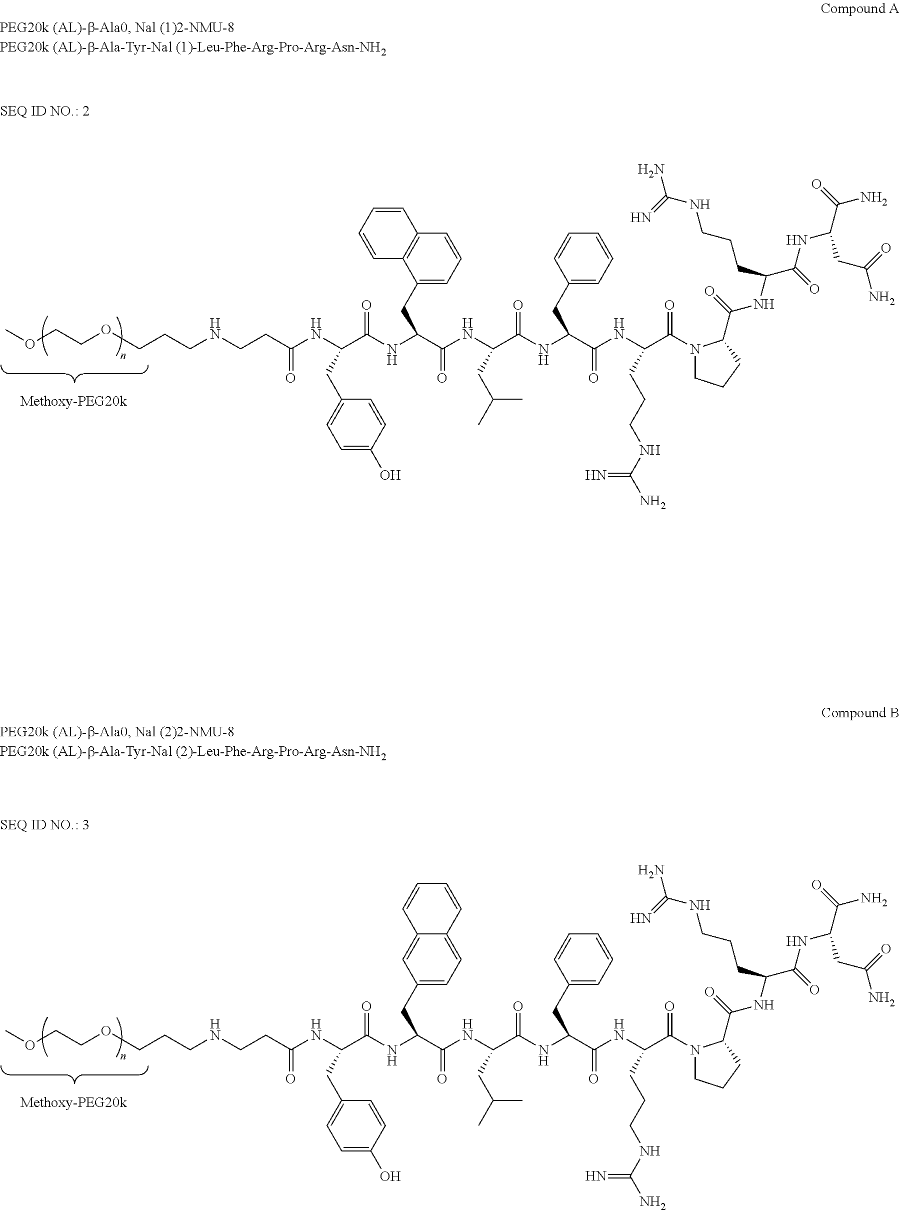

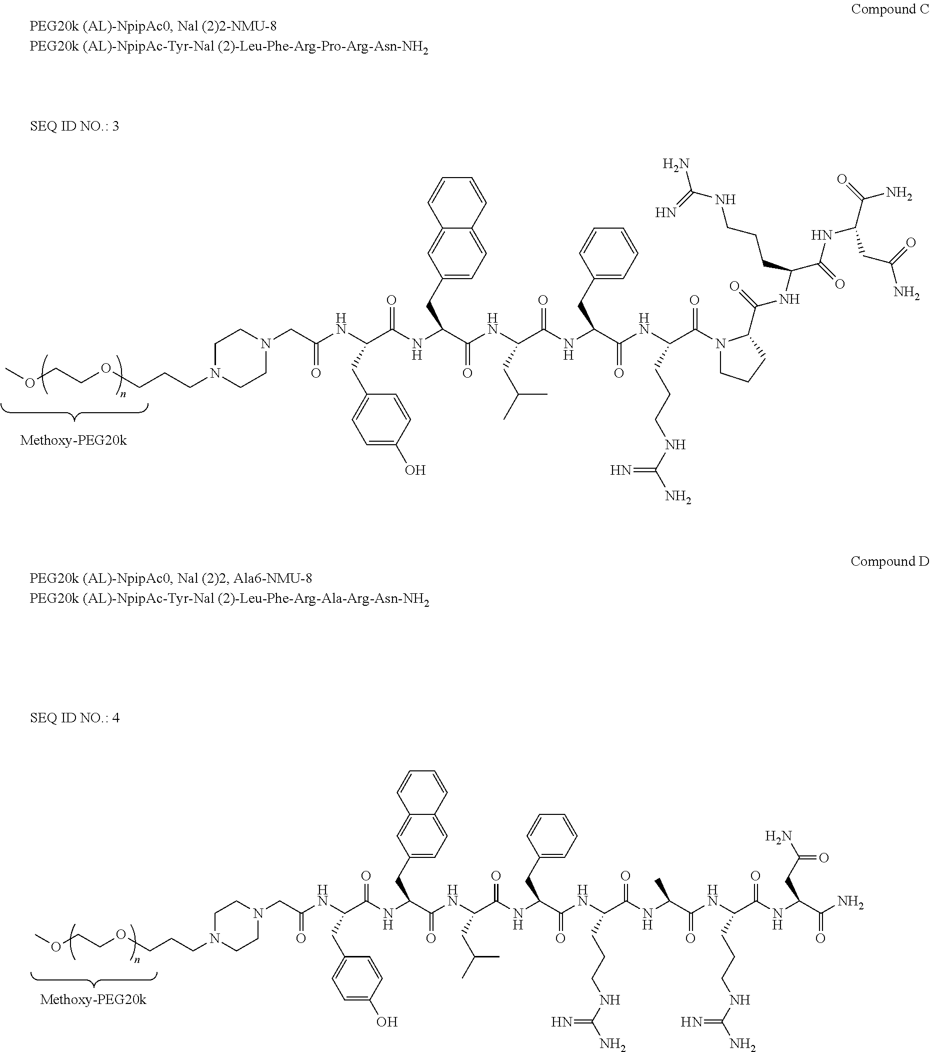

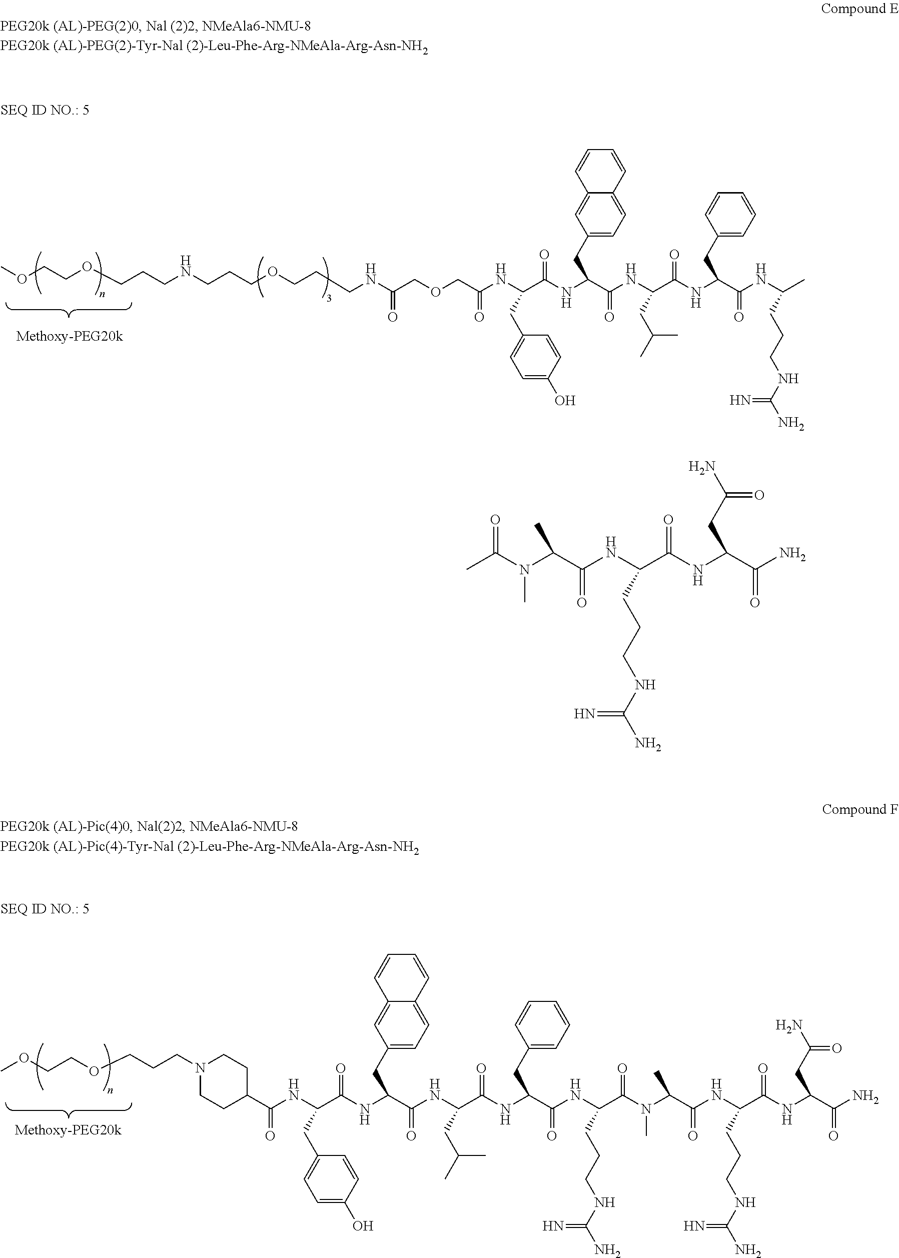

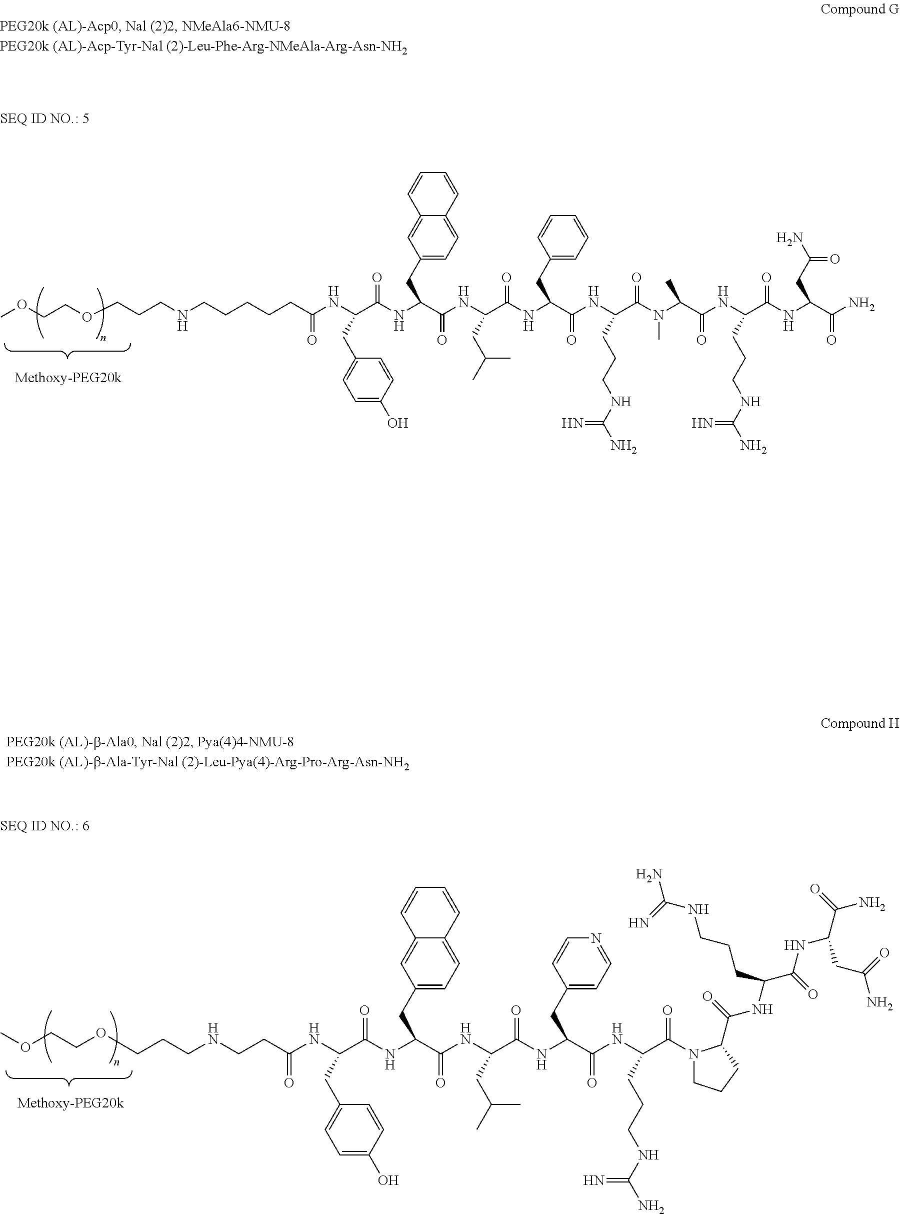

### Reference Example 1

### (Synthetic Method a): Production of β-Ala0,Nal(1)2-NMU-8

### (Compound 1: β-Ala-Tyr-Nal(1)-Leu-Phe-Arg-Pro-Arg-Asn-NH₂)

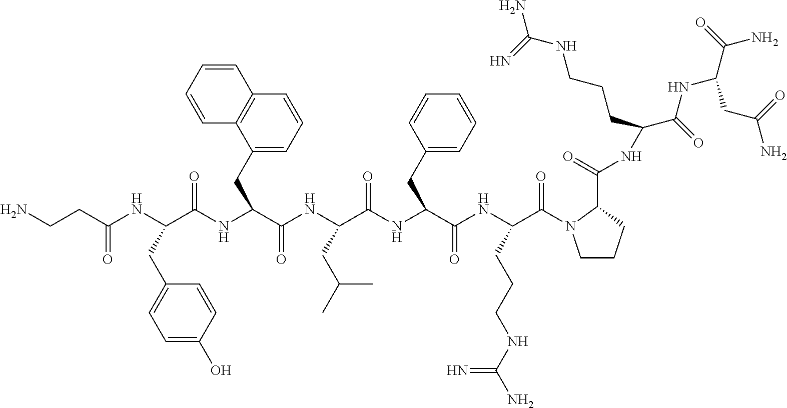

A Sieber amide resin (14.5 mg, 0.01 mmol) was weighed and placed in a reactor, washed with DMF, and stirred in DMF for 20 minutes so as to allow the resin to be swollen. Sequentially, an N-terminal Fmoc group was deprotected by treatment with 20% piperidine/DMF. Then, Fmoc-Asn(Trt)-OH (29.8 mg), 0.5 M HOAt/DMF solution (0.1 mL), and DIPCDI (8.0 μL) were added thereto and treated for 120 minutes, thereby introducing Asn(Trt) residue. Similarly to the above, deprotection of Fmoc group and condensation were repeated to thereby introduce Arg(Pbf), Pro, Arg(Pbf), Phe, Leu, Nal(1), Tyr(tBu), and β-Ala. The N-terminal Fmoc group of the obtained resin was deprotected by treatment with 20% piperidine/DMF. Then, the resulting product was washed with methanol, and dried to yield I-Ala-Tyr(tBu)-Nal(1)-Leu-Phe-Arg(Pbf)-Pro-Arg(Pbf)-Asn(Trt)-Sieber amide resin. The entire amount of the obtained resin was treated with a TFA cocktail (TFA/thioanisole/m-cresol/H₂O/EDT/TIS=80/5/5/5/2.5/2.5, 0.35 mL) for 90 minutes. Thereafter, diethyl ether was added to the reaction mixture, and centrifugation was performed to precipitate the deposited white powder. Then, decantation was repeated twice to remove the diethyl ether. The residue was dissolved in an acetic acid solution, and filtrated through a disc filter with a pore size of 0.45 μm to remove particulates. Thereafter, preparative purification was performed using HPLC (column: Daisopak-SP100-5-ODS-P, 10 mmID×250 mmL, produced by DAISO Co., Ltd.; mobile phase: linear density gradient elution with 0.1% TFA-water/0.1% TFA-containing acetonitrile=82/18 to 52/48, 30 minutes, flow rate: 4 mL/minute). The eluate was fractionated into test tubes, and the elution fractions containing a target product were collected, concentrated, and freeze-dried to obtain the title compound (6.5 mg).

MALDI-TOF/MS: [M+H]⁺ 1232.3 (Calcd. 1232.7)

HPLC elution time: 5.1 minutes

### Elution Conditions

Column: Merck Chromolith Performance RP-18e (4.6 mmID×100 mmL)

Linear density gradient elution (10 minutes) with eluants: 0.1% TFA-water/0.1% TFA-containing acetonitrile=95/5-35/65.

Flow rate: 3.0 mL/minute

### Reference Example 2

### (Synthetic Method b): Production of β-Ala0,Nal(2)2-NMU-8

### (Compound 2: Z-Ala-Tyr-Nal(2)-Leu-Phe-Arg-Pro-Arg-Asn-NH₂)

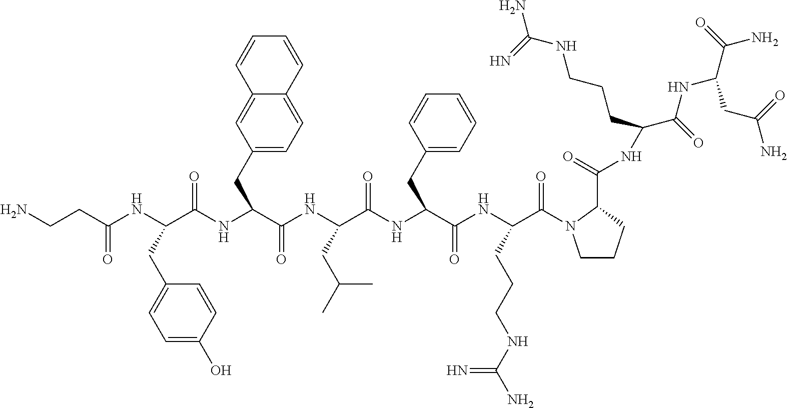

A Sieber amide resin (14.5 mg, 0.01 mmol) was weighed and placed in a reactor, washed with DMF, and stirred in DMF for 20 minutes so as to allow the resin to be swollen. Sequentially, an N-terminal Fmoc group was deprotected by treatment with 20% piperidine/DMF. Then, Fmoc-Asn(Trt)-OH (29.8 mg), 0.5 M HOAt/DMF solution (0.1 mL), and DIPCDI (8.0 μL) were added thereto and treated for 120 minutes, thereby introducing Asn(Trt) residue. Similarly to the above, deprotection of Fmoc group and condensation were repeated to thereby introduce Arg(Pbf), Pro, Arg(Pbf), Phe, Leu, Nal(2), Tyr(tBu), and β-Ala. The N-terminal Fmoc group of the obtained resin was deprotected by treatment with 20% piperidine/DMF. Then, the resulting product was washed with methanol, and dried to yield β-Ala-Tyr(tBu)-Nal(2)-Leu-Phe-Arg(Pbf)-Pro-Arg(Pbf)-Asn(Trt)-Sieber amide resin. The entire amount of the obtained resin was treated with a TFA cocktail (TFA/thioanisole/m-cresol/H₂O/EDT/TIS=80/5/5/5/2.5/2.5, 0.35 mL) for 90 minutes. Thereafter, diethyl ether was added to the reaction mixture, and centrifugation was performed to precipitate the deposited white powder. Then, decantation was repeated twice to remove the diethyl ether. The residue was dissolved in an acetic acid solution, and filtrated through a disc filter with a pore size of 0.45 μm to remove particulates. Thereafter, preparative purification was performed using HPLC (column: Daisopak-SP100-5-ODS-P, 10 mmID×250 mmL, produced by DAISO Co., Ltd.; mobile phase: linear density gradient elution with 0.1% TFA-water/0.1% TFA-containing acetonitrile=82/18 to 52/48, 30 minutes, flow rate: 4 mL/minute). The eluate was fractionated into test tubes, and the elution fractions containing a target product were collected, concentrated, and freeze-dried to obtain the title compound (10.8 mg).

MALDI-TOF/MS: [M+H]⁺ 1232.4 (Calcd. 1232.7)

HPLC elution time: 5.2 minutes

### Elution Conditions

Column: Merck Chromolith Performance RP-18e (4.6 mil)×100 mmL)

Linear density gradient elution (10 minutes) with eluants: 0.1% TFA-water/0.1% TFA-containing acetonitrile=95/5-35/65.

Flow rate: 3.0 mL/minute

### Reference Example 3

### (Synthetic Method c): Production of NpipAc0,Nal(2)2-NMU-8

### (Compound 3: NpipAc-Tyr-Nal(2)-Leu-Phe-Arg-Pro-Arg-Asn-NH₂)

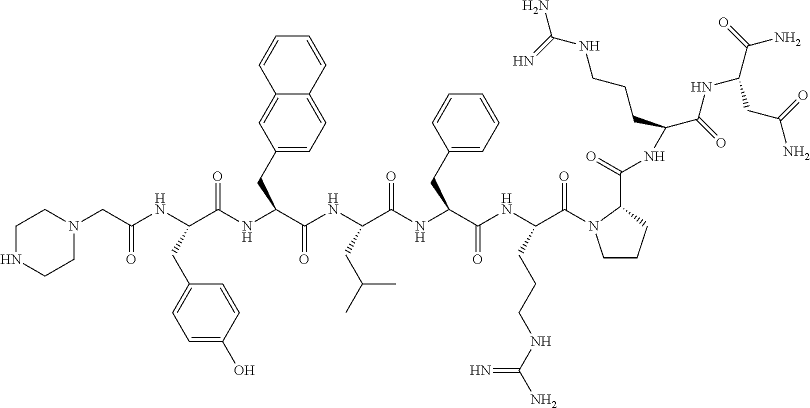

A Sieber amide resin (14.5 mg, 0.01 mmol) was weighed and placed in a reactor, washed with DMF, and stirred in DMF for 20 minutes so as to allow the resin to be swollen. Sequentially, an N-terminal Fmoc group was deprotected by treatment with 20% piperidine/DMF. Then, Fmoc-Asn(Trt)-OH (29.8 mg), 0.5 M HOAt/DMF solution (0.1 mL), and DIPCDI (8.0 μL) were added thereto and treated for 120 minutes, thereby introducing Asn(Trt) residue. Similarly to the above, deprotection of Fmoc group and condensation were repeated to thereby introduce Arg(Pbf), Pro, Arg(Pbf), Phe, Leu, Nal(2), and Tyr(tBu). After the N-terminal Fmoc group of the obtained resin was deprotected by treatment with 20% piperidine/DMF, 2-(1-tert-butoxycarbonylpiperazin-4-yl)acetic acid was condensed in a manner similar to the above, and the obtained resin was washed with methanol, and dried to yield 2-(1-tert-butoxycarbonylpiperazin-4-yl)acetyl-Tyr(tBu)-Nal(2)-Leu-Phe-Arg(Pbf)-Pro-Arg(Pbf)-Asn(Trt)-Sieber amide resin. The entire amount of the obtained resin was treated with a TFA cocktail (TFA/thioanisole/m-cresol/H₂O/EDT/TIS=80/5/5/5/2.5/2.5, 0.35 mL) for 90 minutes. Thereafter, diethyl ether was added to the reaction mixture, and centrifugation was performed to precipitate the deposited white powder. Then, decantation was repeated twice to remove the diethyl ether. The residue was dissolved in an acetic acid solution, and filtrated through a disc filter with a pore size of 0.45 μm to remove particulates. Thereafter, preparative purification was performed using HPLC (column: Daisopak-SP100-5-ODS-P, 10 mmID×250 mmL, produced by DAISO Co., Ltd.; mobile phase: linear density gradient elution with 0.1% TFA-water/0.1% TFA-containing acetonitrile=82/18 to 52/48, 30 minutes, flow rate: 4 mL/minute). The eluate was fractionated into test tubes, and the elution fractions containing a target product were collected, concentrated, and freeze-dried to obtain the title compound (7.9 mg).

MALDI-TOF/MS: [M+H]⁺ 1287.7 (Calcd. 1287.7)

HPLC elution time: 5.2 minutes

### Elution Conditions

Column: Merck Chromolith Performance RP-18e (4.6 mmID×100 mmL)

Linear density gradient elution (10 minutes) with eluants: 0.1% TFA-water/0.1% TFA-containing acetonitrile=95/5-35/65.

Flow rate: 3.0 ml/minute

### Reference Example 4

### (Synthetic Method d): Production of NpipAc0,Nal(2)2,Ala6-NMU-8

### (Compound 4: NpipAc-Tyr-Nal(2)-Leu-Phe-Arg-Ala-Arg-Asn-NH₂)

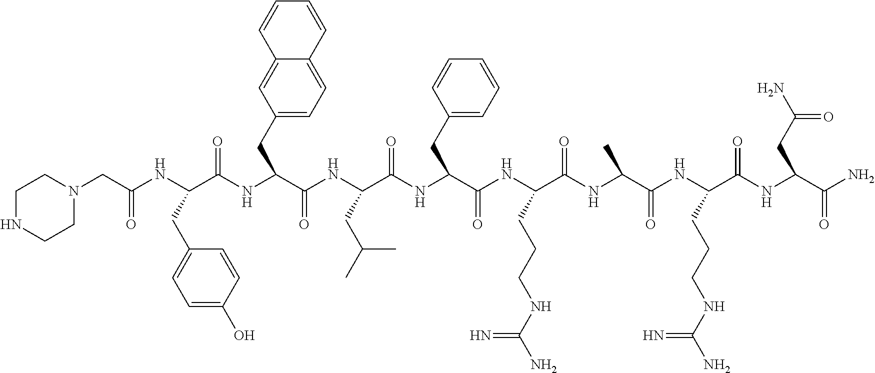

A Sieber amide resin (14.5 mg, 0.01 mmol) was weighed and placed in a reactor, washed with DMF, and stirred in DMF for 20 minutes so as to allow the resin to be swollen. Sequentially, an N-terminal Fmoc group was deprotected by treatment with 20% piperidine/DMF. Then, Fmoc-Asn(Trt)-08 (29.8 mg), 0.5 M HOAt/DMF solution (0.1 mL), and DIPCDI (8.0 μL) were added thereto and treated for 120 minutes, thereby introducing Asn(Trt) residue. Similarly to the above, deprotection of Fmoc group and condensation were repeated to thereby introduce Arg(Pbf), Ala, Arg(Pbf), Phe, Leu, Nal(2), and Tyr(tBu). After the N-terminal Fmoc group of the obtained resin was deprotected by treatment with 20% piperidine/DMF, 2-(1-tert-butoxycarbonylpiperazin-4-yl)acetic acid was condensed in a manner similar to the above, and the obtained resin was washed with methanol, and dried to yield 2-(1-tert-butoxycarbonylpiperazin-4-yl)acetyl-Tyr(tBu)-Nal(2)-Leu-Phe-Arg(Pbf)-Ala-Arg(Pbf)-Asn(Trt)-Sieber amide resin. The entire amount of the obtained resin was treated with a TFA cocktail (TFA/thioanisole/m-cresol/H₂O/EDT/TIS=80/5/5/5/2.5/2.5, 0.35 mL) for 90 minutes. Thereafter, diethyl ether was added to the reaction mixture, and centrifugation was performed to precipitate the deposited white powder. Then, decantation was repeated twice to remove the diethyl ether. The residue was dissolved in an acetic acid solution and filtrated through a disc filter with a pore size of 0.45 μm to remove particulates. Thereafter, preparative purification was performed using HPLC (column: Daisopak-SP100-5-ODS-P, 10 mmID×250 mmL, produced by DAISO Co., Ltd.; mobile phase: linear density gradient elution with 0.1% TFA-water/0.1% TFA-containing acetonitrile=82/18 to 52/48, 30 minutes, flow rate: 4 mL/minute). The eluate was fractionated into test tubes, and the elution fractions containing a target product were collected, concentrated, and freeze-dried to obtain the title compound (9.3 mg).

MALDI-TOF/MS: [M+H]⁺ 1261.5 (Calcd. 1261.7)

HPLC elution time: 5.2 minutes

### Elution Conditions

Column: Merck Chromolith Performance RP-18e (4.6 mmID×100 mmL)

Linear density gradient elution (10 minutes) with eluants: 0.1% TFA-water/0.1% TFA-containing acetonitrile=95/5-35/65.

### Reference Example 5

### (Synthetic Method e): Production of PEG(2)0,Nal(2)2,NMeAla6-NMU-8

### (Compound 5: PEG(2)-Tyr-Nal(2)-Leu-Phe-Arg-NMeAla-Arg-Asn-NH₂)

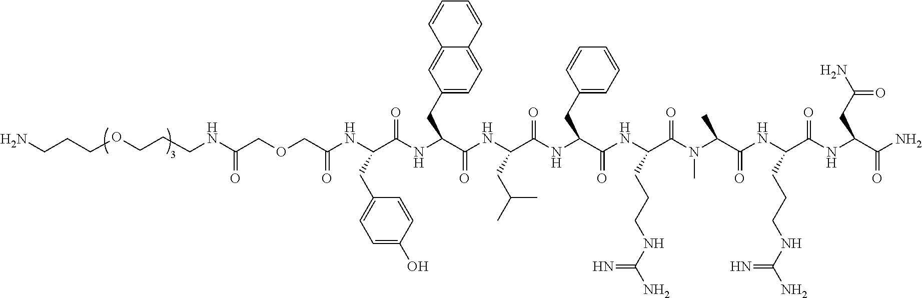

A Sieber amide resin (362.3 mg, 0.25 mmol) was weighed and placed in a reactor, washed with DMF, and stirred in DMF for 20 minutes so as to allow the resin to be swollen. Sequentially, an N-terminal Fmoc group was deprotected by treatment with 20% piperidine/DMF. Then, Fmoc-Asn(Trt)-OH (596.7 mg), 0.5 M HOAt/DMF solution (2.0 mL), and DIPCDI (159.0 μL) were added thereto and treated for 90 minutes, thereby introducing Asn(Trt) residue. Similarly to the above, deprotection of Fmoc group and condensation were repeated to thereby introduce Arg(Pbf), NMeAla, Arg(Pbf), Phe, Leu, Nal(2), and Tyr(tBu). The N-terminal Fmoc group of the obtained resin was deprotected by treatment with 20% piperidine/DMF. Then, the resulting product was washed with methanol and, dried to yield Tyr(tBu)-Nal(2)-Leu-Phe-Arg(Pbf)-NMeAla-Arg(Pbf)-Asn(Trt)-Sieber amide resin.

An amount of 54.3 mg out of the entire amount of the obtained resin was weighed and placed in a reactor, washed with DMF, and stirred in DMF for 20 minutes so as to allow the resin to be swollen. Sequentially, Fmoc-PEG(2)-OH (44.7 mg), 0.5 M HOAt/DMF solution (0.16 mL), and DIPCDI (13.0 μL) were added thereto and treated for 90 minutes, thereby introducing PEG(2) residue. The N-terminal Fmoc group of the obtained resin was deprotected by treatment with 20% piperidine/DMF. Then, the resulting product was washed with methanol, and dried to yield PEG(2)-Tyr(tBu)-Nal(2)-Leu-Phe-Arg(Pbf)-NMeAla-Arg(Pbf)-Asn(Trt)-Sieber amide resin. The entire amount of the obtained resin was treated with a TFA cocktail (TFA/thioanisole/m-cresol/H₂O/EDT/TIS=80/5/5/5/2.5/2.5, 1 ml) for 90 minutes. Thereafter, diethyl ether was added to the reaction mixture, and centrifugation was performed to precipitate the deposited white powder. Then, decantation was repeated twice to remove the diethyl ether. The residue was dissolved in an acetic acid solution and filtrated through a disc filter with a pore size of 0.45 μm to remove particulates. Thereafter, preparative purification was performed using HPLC (column: Daisopak-SP100-5-ODS-P, 20 mmID×250 mmL, produced by DAISO Co., Ltd.; mobile phase: linear density gradient elution with 0.1% TFA-water/0.1% TFA-containing acetonitrile=76/24 to 66/34, 60 minutes, flow rate: 8 mL/minute). The eluate was fractionated into test tubes, and the elution fractions containing a target product were collected, concentrated, and freeze-dried to obtain the title compound (14.5 mg).

MALDI-TOF/MS: [M+H]⁺ 1468.3 (Calcd. 1467.8)

HPLC elution time: 10.0 minutes

### Elution Conditions

Column: Merck Chromolith Performance RP-18e (4.6 mmID×100 mmL)

Linear density gradient elution (25 minutes) with eluants: 0.1% PFA-water/0.1% TFA-containing acetonitrile=80/20-30/70.

Flow rate: 1.0 mL/minute

### Reference Example 6

### (Synthetic Method f): Production of Pic(4)0,Nal(2)2,NMeAla6-NMU-8

### (Compound 6: Pic(4)-Tyr-Nal(2)-Leu-Phe-Arg-NMeAla-Arg-Asn-NH₂)

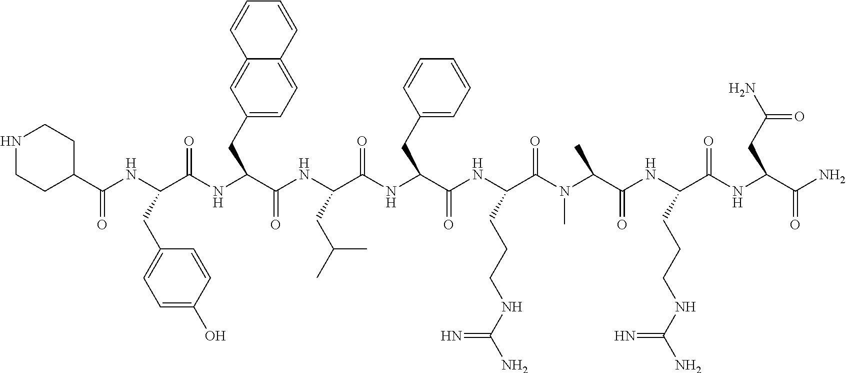

The Tyr(tBu)-Nal(2)-Leu-Phe-Arg(Pbf)-NMeAla-Arg(Pbf)-Asn(Trt)-Sieber amide resin (54.3 mg, 0.02 mmol) obtained in Reference Example 5 was weighed and placed in a reactor, washed with DMF, and stirred in DMF for 20 minutes so as to allow the resin to be swollen. Sequentially, Fmoc-Pic(4)-OH (28.1 mg), 0.5 M HOAt/DMF solution (0.16 mL), and DIPCDI (13.0 μL) were added thereto and treated for 90 minutes, thereby introducing Pic(4) residue. The N-terminal Fmoc group of the obtained resin was deprotected by treatment with 20% piperidine/DMF. Then, the resulting product was washed with methanol, and dried to yield Pic(4)-Tyr(tBu)-Nal(2)-Leu-Phe-Arg(Pbf)-NMeAla-Arg(Pbf)-Asn(Trt)-Sieber amide resin. The entire amount of the obtained resin was treated with a TFA cocktail (TFA/thioanisole/m-cresol/H₂O/EDT/TIS=80/5/5/5/2.5/2.5, 1 mL) for 90 minutes. Thereafter, diethyl ether was added to the reaction mixture, and centrifugation was performed to precipitate the deposited white powder. Then, decantation was repeated twice to remove the diethyl ether. The residue was dissolved in an acetic acid solution and filtrated through a disc filter with a pore size of 0.45 μm to remove particulates. Thereafter, preparative purification was performed using HPLC (column: Daisopak-SP100-5-ODS-P, 20 mmID×250 mmL, produced by DAISO Co., Ltd.; mobile phase: linear density gradient elution with 0.1% TFA-water/0.1% TFA-containing acetonitrile=75/25 to 65/35, 60 minutes, flow rate: 8 mL/minute). The eluate was fractionated into test tubes, and the elution fractions containing a target product were collected, concentrated, and freeze-dried to obtain the title compound (19.0 mg).

MALDI-TOF/MS: [M+H]⁺ 1260.7 (Calcd. 1260.7)

HPLC elution time: 9.1 minutes

### Elution Conditions

Column: Merck Chromolith Performance RP-18e (4.6 μmmID×100 mmL)

Linear density gradient elution (25 minutes) with eluants: 0.1% TFA-water/0.1% TFA-containing acetonitrile=80/20-30/70.

Flow rate: 1.0 mL/minute

### Reference Example 7

### (Synthetic Method g): Production of Acp0,Nal(2)2,NMeAla6-NMU-8

### (Compound 7: Acp-Tyr-Nal(2)-Leu-Phe-Arg-NMeAla-Arg-Asn-NH₂)

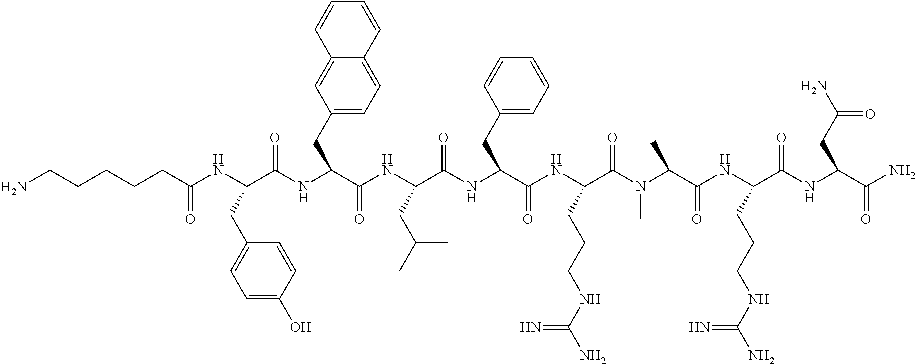

The Tyr(tBu)-Nal(2)-Leu-Phe-Arg(Pbf)-NMeAla-Arg(Pbf)-Asn(Trt)-Sieber amide resin (54.3 mg, 0.02 mmol) obtained in Reference Example 5 was weighed and placed in a reactor, washed with DMF, and stirred in DMF for 20 minutes so as to allow the resin to be swollen. Sequentially, Fmoc-Acp-OH (28.3 mg), 0.5 M HOAt/DMF solution (0.16 mL), and DIPCDI (13.0 μL) were added thereto and treated for 90 minutes, thereby introducing Acp residue. The N-terminal Fmoc group of the obtained resin was deprotected by treatment with 20% piperidine/DMF. Then, the resulting product was washed with methanol, and dried to yield Acp-Tyr(tBu)-Nal(2)-Leu-Phe-Arg(Pbf)-NMeAla-Arg(Pbf)-Asn(Trt)-Sieber amide resin. The entire amount of the obtained resin was treated with a TFA cocktail (TFA/thioanisole/m-cresol/H₂O/EDT/TIS=80/5/5/5/2.5/2.5, 1 mL) for 90 minutes. Thereafter, diethyl ether was added to the reaction mixture, and centrifugation was performed to precipitate the deposited white powder. Then, decantation was repeated twice to remove the diethyl ether. The residue was dissolved in an acetic acid solution and filtrated through a disc filter with a pore size of 0.45 μm to remove particulates. Thereafter, preparative purification was performed using HPLC (column: Daisopak-SP100-5-ODS-P, 20 mmID×250 mmL, produced by DAISO Co., Ltd.; mobile phase: linear density gradient elution with 0.1% TFA-water/0.1% TFA-containing acetonitrile=75/25 to 65/35, 60 minutes, flow rate: 8 mL/minute). The eluate was fractionated into test tubes, and the elution fractions containing a target product were collected, concentrated, and freeze-dried to obtain the title compound (14.2 mg).

MALDI-TOF/MS: [M+H]⁺ 1262.9 (Calcd. 1262.7)

HPLC elution time: 9.5 minutes

### Elution Conditions

Column: Merck Chromolith Performance RP-18e (4.6 mmID×100 mmL)

Linear density gradient elution (25 minutes) with eluants: 0.1% TEA-water/0.1% TFA-containing acetonitrile=80/20-30/70.

Flow rate: 1.0 mL/minute

### Example 1

### (Synthetic Method h): Production of PEG20k(AL)-β-Ala0,Nal(2)2-NMU-8 (Compound B)

SUNBRIGHT ME-200AL (133.9 mg, 6.5 μmol) produced by NOF CORPORATION and Compound 2 (β-Ala-Tyr-Nal(2)-Leu-Phe-Arg-Pro-Arg-Asn-NH₂) (8.0 mg) obtained in Reference Example 2 were dissolved in 1% acetic acid/DMF (10 mL), followed by the addition of sodium triacetoxyborohydride (55.1 mg). The mixture was stirred at room temperature overnight. Thereafter, diethyl ether was added to the reaction mixture and centrifugation was performed to precipitate the deposited white powder. Then, the diethyl ether was removed by decantation. The residue was dissolved in 0.1 M acetic acid solution (150 mL), and SP Sephadex C-50 ion exchange resin (capacity: 45 mL) was added thereto. After the resulting product was left to stand at room temperature for 2 hours, the resin was collected by filtration, and washed sequentially with 0.1 M acetic acid and 10 mM ammonium formate/0.1 M acetic acid. Thereafter, the target product was eluted from the ion exchange resin with 2.0 M ammonium formate/20% acetonitrile, and then with 3.2 M ammonium formate/20% acetonitrile. The obtained eluate was concentrated and filtrated through a disc filter with a pore size of 0.45 μm to remove particulates. Thereafter, preparative purification was performed using HPLC (column: CAPCELL PAY CN UG120 S5, 20 mmID×250 mmL, produced by Shiseido Co., Ltd.; mobile phase: density gradient elution with 0.1% TFA-water/0.1% TFA-containing acetonitrile=95/5 (0 minute)-76/24 (5 minutes)-66/34 (10 minutes)-46/54 (70 minutes), the times in parentheses indicate the times after sample injection; flow rate: 8 mL/minutes). The eluate was fractionated into test tubes, and the elution fractions containing a target product were collected, concentrated, and freeze-dried to yield the title compound (38.0 mg).

MALDI-TOF/MS: measured value: 21051.8-24542.8 (molecular weight: calcd. 21816.4)

HPLC elution time: 16.6 minutes

### Elution Conditions

Column: CAPCELL PAK CN UG120 S5 (4.6 mmID×250 mmL)

Linear density gradient elution (25 minutes) with eluants: 0.1% TFA-water/0.1% TFA-containing acetonitrile=80/20-30/70

Flow rate: 1.0 mL/minute

### Example 2

### (Synthetic Method i): Production of PEG20k(AL)-NpipAc0,Nal(2)2-NMU-8 (Compound C)

SUNBRIGHT ME-200AL (112.1 mg, 5.4 μmol) produced by NOF CORPORATION and Compound 3 (NpipAc-Tyr-Nal(2)-Leu-Phe-Arg-Pro-Arg-Asn-NH₂) (7.0 mg) obtained in Reference Example 3 were dissolved in 1% acetic acid/DMF (6.0 mL), followed by the addition of sodium triacetoxyborohydride (46.1 mg). The mixture was stirred at room temperature overnight. Thereafter, diethyl ether was added to the reaction mixture and centrifugation was performed to precipitate the deposited white powder. Then, the diethyl ether was removed by decantation. The residue was dissolved in 0.1 M acetic acid solution (150 mL), and SP Sephadex C-50 ion exchange resin (capacity: 45 mL) was added thereto. After the resulting product was left to stand at room temperature for 1 hour, the resin was collected by filtration, and washed sequentially with 0.1 M acetic acid and 10 mM ammonium formate/0.1 M acetic acid. Thereafter, the target product was eluted from the ion exchange resin with 2.0 M ammonium formate/20% acetonitrile, and then with 3.2 M ammonium formate/20% acetonitrile. The obtained eluate was concentrated and filtrated through a disc filter with a pore size of 0.45 μm to remove particulates. Thereafter, preparative purification was performed using HPLC (column: CAPCELL PAK CN UG120 S5, 20 mmID×250 mmL, produced by Shiseido Co., Ltd.; mobile phase: density gradient elution with 0.1% TFA-water/0.1% TFA-containing acetonitrile=95/5 (0 minute)-76/24 (5 minutes)-66/34 (10 minutes)-46/54 (70 minutes), the times in parentheses indicate the times after sample injection; flow rate: 8 mL/minutes). The eluate was fractionated into test tubes, and the elution fractions containing a target product were collected, concentrated, and freeze-dried to yield the title compound (33.2 mg).

MALDI-TOF/MS: measured value: 20582.1-24521.5 (molecular weight: calcd. 21871.5)

HPLC elution time: 16.6 minutes

### Elution Conditions

Column: CAPCELL PAK CN UG120 S5 (4.6 mmID×250 mmL)

Linear density gradient elution (25 minutes) with eluants: 0.1% TFA-water/0.1% TFA-containing acetonitrile=80/20-30/70

Flow rate: 1.0 mL/minute

### Example 3

### (Synthetic Method j): Production of PEG20k(AL)-NpipAc0,Nal(2)2,Ala6-NMU-8 (Compound D)

SUNBRIGHT ME-200AL (129.8 mg, 6.3 μmol) produced by NOF CORPORATION and Compound 4 (NpipAc-Tyr-Nal(2)-Leu-Phe-Arg-Ala-Arg-Asn-NH₂) (8.0 mg) obtained in Reference Example 4 were dissolved in 1% acetic acid/DMF (7.0 mL), followed by the addition of sodium triacetoxyborohydride (53.4 mg). The mixture was stirred at room temperature overnight. Thereafter, diethyl ether was added to the reaction mixture and centrifugation was performed to precipitate the deposited white powder. Then, the diethyl ether was removed by decantation. The residue was dissolved in 0.1 M acetic acid solution (150 mL), and SP Sephadex C-50 ion exchange resin (capacity: 45 mL) was added thereto. After the resulting product was left to stand at room temperature for 2 hours, the resin was collected by filtration, and washed sequentially with 0.1 M acetic acid and 10 mM ammonium formate/0.1 M acetic acid. Thereafter, the target product was eluted from the ion exchange resin with 2.0 M ammonium formate/20% acetonitrile, and then with 3.2 M ammonium formate/20% acetonitrile. The obtained eluate was concentrated and filtrated through a disc filter with a pore size of 0.45 μm to remove particulates. Thereafter, preparative purification was performed using HPLC (column: CAPCELL PAK CN UG120 S5, 20 mmID×250 mmL, produced by Shiseido Co., Ltd.; mobile phase: density gradient elution with 0.1% TFA-water/0.1% TFA-containing acetonitrile ˜95/5 (0 minute)-76/24 (5 minutes)-66/34 (10 minutes)-46/54 (70 minutes), the times in parentheses indicate the times after sample injection; flow rate: 8 mL/minutes). The eluate was fractionated into test tubes, and the elution fractions containing a target product were collected, concentrated, and freeze-dried to yield the title compound (54.0 mg).

MALDI-TOF/MS: measured value: 20532.6-24270.9 (molecular weight: calcd. 21845.5)

HPLC elution time: 16.6 minutes

### Elution Conditions

Column: CAPCELL PAK CN UG120 S5 (4.6 mmID×250 mmL)

Linear density gradient elution (25 minutes) with eluants: 0.1% TFA-water/0.1% TFA-containing acetonitrile=80/20-30/70

Flow rate: 1.0 mL/minute

### Example 4

### (Synthetic Method k): Production of PEG20k(AL)-Pic(4)0,Nal(2)2,NMeAla6-NMU-8 (Compound F)

SUNBRIGHT ME-200AL (115.4 mg, 5.6 μmol) produced by NOF CORPORATION and Compound 6 (Pic(4)-Tyr-Nal(2)-Leu-Phe-Arg-NMeAla-Arg-Asn-NH₂) (7.0 mg) obtained in Reference Example 6 were dissolved in 1% acetic acid/DMF (6.0 mL), followed by the addition of sodium triacetoxyborohydride (47.5 mg). The mixture was stirred at room temperature for 3 hours. Thereafter, diethyl ether was added to the reaction mixture and centrifugation was performed to precipitate the deposited white powder. Then, the diethyl ether was removed by decantation. The residue was dissolved in 0.1 M acetic acid solution (150 mL), and SP Sephadex C-50 ion exchange resin (capacity: 45 mL) was added thereto. After the resulting product was left to stand at room temperature for 1 hour, the resin was collected by filtration, and washed sequentially with 0.1 M acetic acid and 10 mM ammonium formate/0.1 M acetic acid. Thereafter, the target product was eluted from the ion exchange resin with 2.0 M ammonium formate/20% acetonitrile, and then with 3.2 M ammonium formate/20% acetonitrile. The obtained eluate was concentrated and filtrated through a disc filter with a pore size of 0.45 μm to remove particulates. Thereafter, preparative purification was performed using HPLC (column: CAPCELL PAK CN UG120 S5, 20 mmID×250 mmL, produced by Shiseido Co., Ltd.; mobile phase: density gradient elution with 0.1% TFA-water/0.1% TFA-containing acetonitrile=95/5 (0 minute)-76/24 (5 minutes)-66/34 (10 minutes)-46/54 (70 minutes), the times in parentheses indicate the times after sample injection; flow rate: 8 mL/minutes). The eluate was fractionated into test tubes, and the elution fractions containing a target product were collected, concentrated, and freeze-dried to yield the title compound (72.9 mg).

MALDI-TOF/MS: measured value: 20175.2-24832.9 (molecular weight: calcd. 21844.5)

HPLC elution time: 17.0 minutes

### Elution Conditions

Column: CAPCELL PAK CN UG120 S5 (4.6 mmID×250 mmL)

Linear density gradient elution (25 minutes) with eluants: 0.1% TFA-water/0.1% TEA-containing acetonitrile=80/20-30/70

Flow rate: 1.0 mL/minute

### Example 5

### (Synthetic Method 1): Production of PEG20k(AL)-Acp0,Nal(2)2,NMeAla6-NMU-8 (Compound G)

SUNBRIGHT ME-200AL (113.3 mg, 5.5 μmol) produced by NOF CORPORATION and Compound 7 (Acp(6)-Tyr-Nal(2)-Leu-Phe-Arg-NMeAla-Arg-Asn-NH₂) (7.0 mg) obtained in Reference Example 7 were dissolved in 1% acetic acid/DMF (6.0 mL), followed by the addition of sodium triacetoxyborohydride (46.6 mg). The mixture was stirred at room temperature for 3 hours. Thereafter, diethyl ether was added to the reaction mixture and centrifugation was performed to precipitate the deposited white powder. Then, the diethyl ether was removed by decantation. The residue was dissolved in 0.1 M acetic acid solution (150 mL), and SP Sephadex C-50 ion exchange resin (capacity: 45 mL) was added thereto. After the resulting product was left to stand at room temperature for 1 hour, the resin was collected by filtration, and washed sequentially with 0.1 M acetic acid and 10 mM ammonium formate/0.1 M acetic acid. Thereafter, the target product was eluted from the ion exchange resin with 2.0 M ammonium formate/20% acetonitrile, and then with 3.2 M ammonium formate/20% acetonitrile. The obtained eluate was concentrated and filtrated through a disc filter with a pore size of 0.45 μm to remove particulates. Thereafter, preparative purification was performed using HPLC (column: CAPCELL PAK CN UG120 S5, 20 mmID×250 mmL, produced by Shiseido Co., Ltd.; mobile phase: density gradient elution with 0.1% TFA-water/0.1% TFA-containing acetonitrile=95/5 (0 minute)-76/24 (5 minutes)-66/34 (10 minutes)-46/54 (70 minutes), the times in parentheses indicate the times after sample injection; flow rate: 8 mL/minutes). The eluate was fractionated into test tubes, and the elution fractions containing a target product were collected, concentrated, and freeze-dried to yield the title compound (31.4 mg).

MALDI-TOF/MS: measured value: 20633.2-24954.1 (molecular weight: calcd. 21846.5)

HPLC elution time: 16.9 minutes

### Elution Conditions

Column: CAPCELL PAK CN UG120 S5 (4.6 mmID×250 mmL)

Linear density gradient elution (25 minutes) with eluants: 0.1% TFA-water/0.1% TFA-containing acetonitrile=80/20-30/70

Flow rate: 1.0 mL/minute

### Example 6

### (Synthetic Method m): Production of PEG20k(AL)-NpipAc0,Nal(2)2-NMU-8 (Compound C)

SUNBRIGHT ME-200AL (1.27 g, 58.3 μmol) produced by NOF CORPORATION and Compound 3 (NpipAc-Tyr-Nal(2)-Leu-Phe-Arg-Pro-Arg-Asn-NH₂) (50.0 mg) obtained in Reference Example 3 were dissolved in Buffer Solution Standard (Phthalate pH Standard Solution) pH 4.01 (25 degrees C.) (19.0 mL), followed by the addition of 2-picoline borane complex (20.8 mg). The mixture was stirred at room temperature for 19 hours. The solution was diluted with 0.1 M acetic acid solution (80 mL), and SP Sephadex C-50 ion exchange resin (capacity: 50 mL) was added thereto. After the resulting product was left to stand at room temperature for 1 hour, the resin was collected by filtration, and washed sequentially with 0.1 M acetic acid and 10 mM ammonium formate/0.1 M acetic acid. Thereafter, the target product was eluted from the ion exchange resin with 2.0 M ammonium formate/20% acetonitrile. The obtained eluate was concentrated and filtrated through a disc filter with a pore size of 0.45 μm to remove particulates. Thereafter, preparative purification was performed using HPLC three times (column: CAPCELL PAK CN UG120 S5, 20 mmID×250 mmL, produced by Shiseido Co., Ltd.; mobile phase: density gradient elution with 0.1% TFA-water/0.1% TFA-containing acetonitrile=95/5 (0 minute)-77/23 (5 minutes)-67/33 (10 minutes)-47/53 (70 minutes), the times in parentheses indicate the times after sample injection; flow rate: 8 mL/minutes). The eluate was fractionated into test tubes, and the elution fractions containing a target product were collected, concentrated, and freeze-dried to yield the title compound (556.1 mg).

MALDI-TOF/MS: measured value: 20000-25000 (molecular weight: calcd. 23086.5)

HPLC elution time: 20.8 minutes

### Elution Conditions

Column: CAPCELL PAK C1 UG120 S5 (4.6 mmID×250 mmL)

Linear density gradient elution (25 minutes) with eluants: 0.1% TFA-water/0.1% TFA-containing acetonitrile=80/20-30/70

Flow rate: 1.0 mL/minute

Table 1 below shows the structure, physicochemical properties, etc., of the compounds synthesized in Reference Examples 1 to 7 and Examples 1 to 5, and compounds synthesized by methods that are similar to the methods of Reference Examples 1 to 7 and Examples 1 to 5. The column titled “Synthetic Method” in the table shows that Compounds 1 to 8, B, C, D, F, and G were synthesized by the synthetic method a, b, c, d, e, f, g, h, i, j, or k; or that the compounds can be synthesized by the synthetic method shown in the column. The column also shows that Compounds A, E, and H were synthesized in a manner similar to the methods shown therein.

The column titled “HPLC (min.)” in the table shows the retention time at which Compounds 1 to 8, B, C, D, F, and G were eluted under the respective elution conditions, and the retention time at which Compounds A, E, and H were eluted under elution conditions similar to those employed in Reference Examples 1 to 7 and Examples 1 to 5.

TABLE 1

| Compound | | | M + H⁺ | HPLC | Synthesized | Analysis |
|---|---|---|---|---|---|---|
| Number | Structure | M + H⁺ (obs.) | (cal.) | (min.) | Method | Conditions |
| 1 | β-Ala0,Nal(1)2-NMU-8 | 1232.3 | 1232.7 | 5.1 | a | m |
| 2 | β-Ala0,Nal(2)2-NMU-8 | 1232.4 | 1232.7 | 5.2 | b | m |
| 3 | NpipAc0,Nal(2)2-NMU-8 | 1287.7 | 1287.7 | 5.2 | c | m |
| 4 | NpipAc0,Nal(2)2,Ala6-NMU-8 | 1261.5 | 1261.7 | 5.2 | d | m |
| 5 | PEG(2)0,Nal(2)2,NMeAla6-NMU-8 | 1468.3 | 1467.8 | 10.0 | e | n |
| 6 | Pic(4)0,Nal(2)2,NMeAla6-NMU-8 | 1260.7 | 1260.7 | 9.1 | f | n |
| 7 | Acp0,Nal(2)2,NMeAla6-NMU-8 | 1262.9 | 1262.7 | 9.5 | g | n |
| 8 | β-Ala0,Nal(2)2,Pya(4)4-NMU-8 | 1233.1 | 1233.7 | 4.4 | a | m |

| Compound | | | NW | HPLC | Synthesized | Analysis |
|---|---|---|---|---|---|---|
| Number | Structure | MALDI MS (obs.) | (cal.) | (min.) | Method | Conditions |
| A | PEG20k(AL)-β-Ala0,Nal(1)2-NMU-8 | 20516.6-24330.1 | 21816.4 | 16.7 | h | o |
| B | PEG20k(AL)-β-Ala0,Nal(2)2-NMU-8 | 21051.8-24542.8 | 21816.4 | 16.6 | h | o |
| C | PEG20k(AL)-NpipAc0,Nal(2)2-NMU-8 | 20582.1-24521.5 | 21871.5 | 16.6 | i | o |
| D | PEG20k(AL)- | 20532.6-24270.9 | 21845.5 | 16.6 | j | o |
| | NpipAc0,Nal(2)2,Ala6-NMU-8 | | | | | |
| E | PEG20k(AL)- | 20829.2-24692.4 | 22051.7 | 17.1 | h | o |
| | PEG(2)0,Nal(2)2,NMeAla6-NMU-8 | | | | | |
| F | PEG20k(AL)- | 20175.2-24832.9 | 21844.5 | 17.0 | k | o |
| | Pic(4)0,Nal(2)2,NMeAla6-NMU-8 | | | | | |
| G | PEG20k(AL)- | 20633.2-24954.1 | 21846.5 | 16.9 | l | o |
| | Acp0,Nal(2)2,NMeAla6-NMU-8 | | | | | |
| H | PEG20k(AL)-β- | 20460.6-24505.0 | 22760.4 | 16.6 | h | o |
| | Ala0,Nal(2)2,Pya(4)4-NMU-8 | | | | | |

m: Merck Chromolith Performance RP-18e, 4.6 × 100 mm, gradient: 5-65% B (A: DW/0.1% TFA; B: 100% AcCN/0.1% TFA), 0 to 10 min., 3 mL/min
n: Merck Chromolith Performance RP-18e, 4.6 × 100 mm, gradient: 20-70% B (A: DW/0.1% TFA; B: 100% AcCN/0.1% TFA), 0-25 min., 1 mL/min
o: CAPCELL PAK CN UG120 S5, 4.6 × 250 mm; gradient: 20-70% B (A: DW/0.1% TFA; B: 100% AcCN/0.1% TFA), 0 to 25 min., 1 mL/min

### Test Example 1

### Ca Influx Assay Using CHO Cells Expressing Human NMUR1 or NMUR2

CHO cells stably expressing human NMUR1 or NMUR2 (J Biol Chem 275(28), pp. 21068-21074 (2000); and J Biol Chem 275(38), pp. 29528-29532 (2000)) were seeded onto a 384-well black/clear plate (Becton Dickinson) (10,000 cells per well) using nucleic acid-free MEM-α medium (Nikken Bio Medical Laboratory) containing 10% dialyzed blood serum (Gemini Bio Products) and 50 μg/mL gentamycin (Invitrogen), and cultured overnight in 5% carbon dioxide at 37° C. Then, after removal of the medium, the cells were loaded with Calcium Kit-Fluo 4 (Dojindo) containing 0.1% fatty acid-free BSA (Wako) at 37° C. (for 15 minutes). Further, test compounds at each concentration, as well as 1 μmol/L porcine NMU-8 (BACHEM) as a control group, were added to the cells, and an elevation of intracellular Ca concentration was monitored for 3 minutes using a FLIPR Tetra system (Molecular Devices). The agonist activity (%) of each of the test compounds for NMUR was calculated using the following formula:

[(W−X)/(Y−X)]×100

wherein W represents a fluorescence value based on the intracellular Ca concentration in cells to which each test compound was added; X represents a fluorescence value based on the intracellular Ca concentration in cells to which only 0.1% DMSO was added; and Y represents a fluorescence value based on the intracellular Ca concentration in cells to which 1 μM porcine NMU-8 was added. The EC₅₀ value of each test compound was calculated using Prism 5 (GraphPad). Table 2 shows the results.

TABLE 2

| Compound | | hNMUR1 | hNMUR2 |
|---|---|---|---|
| Number | Structure | EC₅₀ | EC₅₀ |
| A | PEG20k(AL)-β-Ala0,Nal(1)2-NMU-8 | >1.0E−06 | 1.0E−08 |
| B | PEG20k(AL)-β-Ala0,Nal(2)2-NMU-8 | 2.1E−07 | 4.5E−09 |
| C | PEG20k(AL)-NpipAc0,Nal(2)2-NMU-8 | 2.3E−07 | 5.6E−09 |
| D | PEG20k(AL)-NpipAc0,Nal(2)2,Ala6-NMU-8 | >1.0E−06 | 8.8E−09 |
| E | PEG20k(AL)-PEG(2)0,Nal(2)2,NMeAla6-NMU-8 | >1.0E−06 | 3.9E−08 |
| F | PEG20k(AL)-Pic(4)0,Nal(2)2,NMeAla6-NMU-8 | >1.0E−06 | 5.4E−08 |
| G | PEG20k(AL)-Acp0,Nal(2)2,NMeAla6-NMU-8 | 1.7E−07 | 5.9E−09 |
| H | PEG20k(AL)-β-Ala0,Nal(2)2,Pya(4)4-NMU-8 | >1.0E−06 | 4.5E−08 |

Table 2 shows that the compounds of the present invention exhibit excellent agonistic activity on NMUR.

### Test Example 2

### Subcutaneous Administration Test in Mice

Anorectic activity of the test compounds was evaluated by the following method.

Each of the test compounds was dissolved in a solvent (physiological saline), and an amount of 600 nmol/kg/day was used. An amount of 2 mL/Kg of an administration liquid obtained by dissolving each test compound in physiological saline was subcutaneously administered to the back of 9- to 10-week-old male C57BL/6J mice (CLEA Japan, Inc.) (at 25° C., having free access to food and water, 12 hours of light period and 12 hours of dark period). After the administration, the mice were returned to breeding cages (individual feeding), pre-weighed food was given, and the food intake 1 day after the initiation of the administration was measured. The food intake was calculated by subtracting the weight of the food remaining from the weight of the food given on the day the administration was initiated. Based on the calculated food intake, the anorectic ratio (%) of each test compound was calculated using the following formula. Table 3 shows the results.

The group where only the solvent was administered as described above was used as a control group.

### Food Intake Inhibition (%):

{(food intake of the control group−food intake of a test compound administration group)/food intake of the control group}×100

TABLE 3

| Compound | | Food intake |
|---|---|---|
| Number | Structure | Inhibition |
| A | PEG20k(AL)-β-Ala0,Nal(1)2-NMU-8 | 64.5 |
| B | PEG20k(AL)-β-Ala0,Nal(2)2-NMU-8 | 75.9 |
| C | PEG20k(AL)-NpipAc0,Nal(2)2-NMU-8 | 88.4 |
| D | PEG20k(AL)-NpipAc0,Nal(2)2,Ala6- | 70.5 |
| | NMU-8 | |
| E | PEG20k(AL)-PEG(2)0,Nal(2)2,NMeAla6- | 68.6 |
| | NMU-8 | |
| F | PEG20k(AL)-Pic(4)0,Nal(2)2,NMeAla6- | 74.3 |
| | NMU-8 | |
| G | PEG20k(AL)-Acp0,Nal(2)2,NMeAla6- | 71.4 |
| | NMU-8 | |

Table 3 shows that the compounds of the present invention exhibit an excellent anorectic activity.

### Test Example 3

### Single Subcutaneous Administration Test in Monkeys

The emetic response induced by the test compounds was evaluated by the following method.

The test compound C was dissolved in physiological saline at a concentration of 1,000 nmol/kg and the test compound D was dissolved in physiological saline at a concentration of 300 nmol/kg, and used separately. An administration liquid obtained by dissolving each test compound in physiological saline was subcutaneously administered to the back of cynomolgus monkeys (2 males and 2 females each, 3 years and 11 months old to 4 years and 4 months old). After the administration, the animals were immediately returned to their individual cages, and videotaped for 10 hours. Vomiting and dry heaves observed during the videotaping were counted. Vomits were counted as the number of instances of vomiting up to 24 hours after the completion of the videotaping. Table 4 shows the results.

TABLE 4

| | No. of vomiting | |
|---|---|---|
| Dose (nmol/kg) | (0-24 h) | |
| Compound C | 0 | n = 4 |
| 1000 nmol/kg | | (2 males, 2 females) |
| Compound D | 0 | n = 4 |
| 300 nmol/kg | | (2 males, 2 females) |

Table 4 shows that the test compounds C and D prevent vomiting.

### Formulation Example 1

### Production of Tablet

| | |
|---|---|
| (1) Compound A | 10.0 mg |
| (2) Lactose | 70.0 mg |
| (3) Corn starch | 50.0 mg |
| (4) Soluble starch | 7.0 mg |
| (5) Magnesium stearate | 3.0 mg |

Using 0.07 ml of an aqueous soluble starch solution (7.0 mg as soluble starch), 10.0 mg of the compound A and 3.0 mg of magnesium stearate are granulated, dried, and mixed with 70.0 mg of lactose and 50.0 mg of corn starch. The mixture is compressed to obtain tablets.

### Formulation Example 2

### Production of Injectable Solution

| | | |
|---|---|---|
| 1) Compound A | 5.0 | mg |
| 2) salt | 20.0 | mg |
| 3) distillated water | ad. 2.0 | ml |

A solution of 5.0 mg of the compound A and 20.0 mg of salt in distilled water is prepared, and water is added thereto to make up the total amount to 2.0 ml. The solution is filtered, and filled in a 2 ml ample under aseptic conditions. The ample is sterilized, and sealed to obtain an injectable solution.

### INDUSTRIAL APPLICABILITY

The compounds of the present invention can be used as an agent for preventing or treating obesity, or as an anorectic agent.

### SEQUENCE LISTING FREE TEXT

### [SEQ ID NO.: 1]

Position 8 is amidated.

### [SEQ ID NO.: 2]

A variant of NMU-8.
Position 2 is 1-naphthylalanine.
Position 8 is amidated.

### [SEQ ID NO.: 3]

A variant of NMU-8.
Position 2 is 2-naphthylalanine.
Position 8 is amidated.

### [SEQ ID NO.: 4]

A variant of NMU-8.
Position 2 is 2-naphthylalanine.
Position 6 is N^{a}-methylalanine.
Position 8 is amidated.

### [SEQ ID NO.: 5]

A variant of NMU-8.
Position 2 is 2-naphthylalanine.
Position 6 is N^{α}-methylalanine.
Position 8 is amidated.

### [SEQ ID NO.: 6]

A variant of NMU-8.
Position 2 is 2-naphthylalanine.
Position 4 is 4-pyridylalanine.
Position 8 is amidated.

### SEQUENCE LISTING

1. A peptide derivative selected from the group consisting of
PEG20k(AL)-β-Ala-Tyr-Nal(1)-Leu-Phe-Arg-Pro-Arg-Asn-NH₂,
PEG20k(AL)-β-Ala-Tyr-Nal(2)-Leu-Phe-Arg-Pro-Arg-Asn-NH₂,
PEG20k(AL)-NpipAc-Tyr-Nal(2)-Leu-Phe-Arg-Pro-Arg-Asn-NH₂,
PEG20k(AL)-NpipAc-Tyr-Nal(2)-Leu-Phe-Arg-Ala-Arg-Asn-NH₂,
PEG20k(AL)-PEG(2)-Tyr-Nal(2)-Leu-Phe-Arg-NMeAla-Arg-Ash-NH₂,
PEG20k(AL)-Pic(4)-Tyr-Nal(2)-Leu-Phe-Arg-NMeAla-Arg-Asn-NH₂,
PEG20k(AL)-Acp-Tyr-Nal(2)-Leu-Phe-Arg-NMeAla-Arg-Asn-NH₂, and
PEG20k(AL)-β-Ala-Tyr-Nal(2)-Leu-Pya(4)-Arg-Pro-Arg-Asn-NH₂;
or a salt thereof.

2. PEG20k(AL)-NpipAc-Tyr-Nal(2)-Leu-Phe-Arg-Pro-Arg-Asn-NH₂, or a salt thereof.

3. PEG20k(AL)-NpipAc-Tyr-Nal(2)-Leu-Phe-Arg-Ala-Arg-Asn-NH₂, or a salt thereof.

4. PEG20k(AL)-Pic(4)-Tyr-Nal(2)-Leu-Phe-Arg-NMeAla-Arg-Asn-NH₂, or a salt thereof.

5. A medicament comprising the peptide derivative or a salt thereof of claim 1.

6. The medicament according to claim 5, which is a neuromedin U receptor agonist.

7. The medicament according to claim 5, which is an anorectic agent.

8. The medicament according to claim 5, which is an agent for preventing or treating obesity.

9. A method for preventing or treating obesity, comprising administering to a mammal an effective amount of the peptide derivative or a salt thereof of claim 1.

10. A method for activating a neuromedin U receptor in a mammal, comprising administering to the mammal an effective amount of the peptide derivative or a salt thereof of claim 1.

11. A method for attenuating food intake in a mammal, comprising administering to the mammal an effective amount of the peptide derivative or a salt thereof of claim 1.

12. Use of the peptide derivative or a salt thereof of claim 1 for producing an agent for preventing or treating obesity.

13. Use of the peptide derivative or a salt thereof of claim 1 for producing an anorectic agent.

14. The peptide derivative or a salt thereof of claim 1 for preventing or treating obesity.

15. The peptide derivative or a salt thereof of claim 1 for attenuating food intake.
